(19) United States
(12) Patent Application Publication
(10) Pub. No.: US 2021/0404994 A1
(43) Pub. Date: Dec. 30, 2021

(54) HEATERS IN CAPACITIVE MICROMACHINED ULTRASONIC TRANSDUCERS AND METHODS OF FORMING AND ACTIVATING SUCH HEATERS

(71) Applicants: Victor L. Pushparaj, Milpitas, CA (US); Nikhil Apte, San Jose, CA (US)

(72) Inventors: Victor L. Pushparaj, Milpitas, CA (US); Nikhil Apte, San Jose, CA (US)

(73) Assignee: Butterfly Network, Inc., Guilford, CT (US)

(21) Appl. No.: 17/355,577

(22) Filed: Jun. 23, 2021

Related U.S. Application Data
(60) Provisional application No. 63/046,658, filed on Jun. 30, 2020.

(51) Int. Cl. G01N 29/24; B06B 1/02; G01N 29/06

(52) U.S. Cl. CPC G01N 29/2406; B06B 1/0292; G01N 2291/26; G01N 29/2431; G01N 29/0654

(57) ABSTRACT

Aspects of the technology described herein relate to a capacitive micromachined ultrasonic transducer (CMUT) and a heater disposed in the CMUT, and forming a capacitive micromachined ultrasonic transducer (CMUT) and a heater disposed in the CMUT. A voltage may be applied to a heater disposed in a CMUT in an ultrasound imaging device in order to cause the heater to generate heat. Based on determining that the collapse voltage of the CMUT has increased by at least a threshold voltage between two times, a voltage may be automatically applied to a heater in the CMUT such that the heater generates heat.

### CROSS-REFERENCE TO RELATED APPLICATIONS

This application claims the benefit of U.S. Provisional Application Ser. No. 63/046,658, filed on Jun. 30, 2020, under Attorney Docket No. B1348.70188US00 and entitled “HEATERS IN CAPACITIVE MICROMACHINED ULTRASONIC TRANSDUCERS AND METHODS OF FORMING AND ACTIVATING SUCH HEATERS”, which is hereby incorporated by reference herein in its entirety.

### FIELD

Generally, the aspects of the technology described herein relate to capacitive micromachined ultrasonic transducers. Some aspects relate to heaters in cavities of capacitive micromachined ultrasonic transducers and methods of forming and activating such heaters.

### BACKGROUND

Ultrasound imaging devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound imaging devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to an aspect of the application, an apparatus includes a capacitive micromachined ultrasonic transducer (CMUT) and a heater disposed in the CMUT.

In some embodiments, the CMUT includes a membrane and an electrode, and the heater is disposed between the membrane and the electrode. In some embodiments, the heater includes a planar resistive layer. In some embodiments, the heater includes a thin film layer. In some embodiments, the heater includes a layer of nichrome, chromium-silicon, or a ferritic iron-chromium-aluminum alloy. In some embodiments, a thickness of the heater is between or equal to approximately 500-3000 angstroms. In some embodiments, a thickness of the heater is between or equal to approximately 500-1500 angstroms.

In some embodiments, the CMUT includes a cavity having a top and a bottom, and the heater is disposed at the top of the cavity. In some embodiments, the CMUT includes a membrane, the membrane includes a silicon layer and an oxide layer, and the heater is disposed on the oxide layer.

In some embodiments, the CMUT further includes a cavity and the heater is disposed adjacent to the cavity. In some embodiments, the CMUT includes a cavity having a top and a bottom, and the heater is disposed at the bottom of the cavity. In some embodiments, the CMUT further includes an electrode and one or more oxide layers disposed on the electrode, and the heater is disposed on one of the oxide layers. In some embodiments, the CMUT further includes a cavity and the heater is disposed adjacent to the cavity. In some embodiments, the one or more oxide layers include a silicon oxide layer and an aluminum oxide layer, the silicon oxide layer is disposed on the electrode, the aluminum oxide layer is disposed on the silicon oxide layer, and the heater is disposed on the aluminum oxide layer. In some embodiments, the CMUT further includes a cavity and the heater is disposed adjacent to the cavity. In some embodiments, the CMUT further includes an electrode and two or more oxide layers disposed on the electrode, and the heater is disposed between two of the two or more oxide layers. In some embodiments, the two or more oxide layers include a silicon oxide layer formed using chemical vapor deposition and a silicon oxide layer formed using high-density plasma chemical-vapor deposition; the silicon oxide layer formed using chemical vapor deposition is disposed on the electrode; the heater is disposed on the silicon oxide layer formed using chemical vapor deposition, and the silicon oxide layer formed using high-density plasma chemical-vapor deposition is disposed on the heater. In some embodiments, the CMUT further includes a cavity and the silicon oxide layer formed using high-density plasma chemical-vapor deposition is disposed adjacent to the cavity. In some embodiments, the CMUT includes an oxide layer and the heater is disposed on the oxide layer.

In some embodiments, the heater is formed using sputtering or chemical vapor deposition. In some embodiments, the heater is laid out in a shape that includes curved lines. In some embodiments, the heater is laid out in a shape that includes lines at right angles. In some embodiments, the heater is laid out in a shape that includes acute angles. In some embodiments, the heater is capable of heating to a temperature approximately equal to or between 20 to 250 degrees Celsius.

In some embodiments, the CMUT includes a first electrode, a second electrode, and one or more oxide layers disposed on the first electrode, and the heater is disposed on the one or more oxide layers and on the second electrode. In some embodiments, the heater is electrically coupled to the second electrode.

In some embodiments, the CMUT is disposed on a substrate including integrated circuitry, and the integrated circuitry is electrically coupled to the second electrode. In some embodiments, the integrated circuitry is configured to apply a voltage to the heater through the second electrode. In some embodiments, the integrated circuitry includes transmit circuitry, receive circuitry, timing and control circuitry, and/or signal conditioning/processing circuitry. In some embodiments, the substrate includes a semiconductor chip. In some embodiments, the CMUT includes a membrane on which the heater is disposed, a first electrical contact disposed on the membrane and electrically coupled to the membrane, and a second electrical contact disposed on the membrane and electrically coupled to the heater. In some embodiments, the CMUT is disposed on a substrate including integrated circuitry, the integrated circuitry is electrically coupled to the second electrical contact, and the integrated circuitry is configured to apply a voltage to the heater through the second electrical contact. In some embodiments, the apparatus further includes a DC-DC converter electrically coupled to the second electrical contact and configured to apply a voltage to the heater through the second electrical contact. In some embodiments, the CMUT and the heater disposed therein are disposed on a substrate including integrated circuitry. In some embodiments, the substrate includes a semiconductor chip. In some embodiments, the integrated circuitry includes transmit circuitry, receive circuitry, timing and control circuitry, and/or signal conditioning/processing circuitry.

In some embodiments, the CMUT and the heater disposed therein are disposed in a handheld ultrasound probe. In some embodiments, the CMUT and the heater disposed therein are disposed in a wearable ultrasound patch. In some embodiments, the CMUT and the heater disposed therein are disposed in an ingestible ultrasound pill.

According to another aspect of the application, a method includes forming a capacitive micromachined ultrasonic transducer (CMUT) and a heater disposed in the CMUT. In some embodiments, the CMUT includes a membrane and an electrode, and forming the CMUT and the heater disposed in the CMUT includes forming the heater between the membrane and the electrode of the CMUT. In some embodiments, the heater includes a planar resistive layer. In some embodiments, the heater includes a thin film layer. In some embodiments, the heater includes a layer of nichrome, chromium-silicon, or a ferritic iron-chromium-aluminum alloy. In some embodiments, a thickness of the heater is between or equal to approximately 500-3000 angstroms. In some embodiments, a thickness of the heater is between or equal to approximately 500-1500 angstroms.

In some embodiments, the CMUT includes a cavity having a top and a bottom, and forming the CMUT and the heater disposed in the CMUT includes forming the heater at the top of the cavity. In some embodiments, forming the CMUT and the heater disposed in the CMUT includes forming an oxide layer on a first substrate, forming a heater on the oxide layer, and forming a cavity on a second substrate and sealing the cavity with the first substrate such that the heater is in the cavity, and wherein the first substrate includes a membrane on the CMUT. In some embodiments, sealing the cavity with the first substrate such that the heater is in the cavity includes sealing the cavity with the first substrate such that the heater is adjacent to the cavity.

In some embodiments, the CMUT includes a cavity having a top and a bottom, and forming the CMUT and the heater disposed in the CMUT includes forming the heater at the bottom of the cavity. In some embodiments, forming the CMUT and the heater disposed in the CMUT includes forming an electrode on a substrate, forming one or more oxide layers on the electrode, forming a heater on the one or more oxide layers, and forming and sealing a cavity on the substrate such that the heater is in the cavity. In some embodiments, sealing the cavity on the substrate such that the heater is in the cavity includes sealing the cavity on the substrate such that the heater is adjacent to the cavity. In some embodiments, forming the one or more oxide layers and forming the heater on the one or more oxide layers include forming a silicon oxide layer on the electrode, forming an aluminum oxide layer on the silicon oxide layer, and forming the heater on the aluminum oxide layer. In some embodiments, sealing the cavity on the substrate such that the heater is in the cavity includes sealing the cavity on the substrate such that the heater is adjacent to the cavity.

In some embodiments, forming the CMUT and the heater disposed in the CMUT includes forming an electrode on a substrate, forming a first oxide layer on the electrode, forming the heater on the first oxide layer, forming a second oxide layer on the heater, and forming and sealing a cavity on the substrate such that the heater is disposed in the CMUT. In some embodiments, forming the first oxide layer includes forming a silicon oxide layer using chemical vapor deposition and forming the second oxide layer includes forming a silicon oxide layer using high-density plasma chemical-vapor deposition. In some embodiments, sealing the cavity on the substrate includes sealing the cavity on the substate such that the second oxide layer is adjacent to the cavity. In some embodiments, forming the CMUT and the heater disposed in the CMUT includes forming an oxide layer in the CMUT and forming the heater on the oxide layer.

In some embodiments, forming the CMUT and the heater disposed in the CMUT includes forming the heater using sputtering or chemical vapor deposition. In some embodiments, forming the CMUT and the heater disposed in the CMUT includes forming the heater in a shape that includes curved lines. In some embodiments, forming the CMUT and the heater disposed in the CMUT includes forming the heater in a shape that includes lines at right angles. In some embodiments, forming the CMUT and the heater disposed in the CMUT includes forming the heater in a shape that includes acute angles. In some embodiments, the heater is capable of heating to a temperature approximately equal to or between 20 to 250 degrees Celsius. In some embodiments, forming the CMUT and the heater disposed in the CMUT includes forming a first electrode and a second electrode on a substrate, forming one or more oxide layers on the first electrode, and forming the heater on the one or more oxide layers and on the second electrode such that the heater is electrically coupled to the second electrode.

In some embodiments, the substrate includes integrated circuitry, and the integrated circuitry is electrically coupled to the second electrode. In some embodiments, the substrate includes a semiconductor chip. In some embodiments, the integrated circuitry includes transmit circuitry, receive circuitry, timing and control circuitry, and/or signal conditioning/processing circuitry. In some embodiments, forming the CMUT and the heater disposed in the CMUT includes forming the heater on a membrane of the CMUT, forming a first contact that is electrically coupled to the membrane, and forming a second contact that is electrically coupled to the heater. In some embodiments, forming the CMUT and the heater disposed in the CMUT includes forming the CMUT on a substrate including integrated circuitry, and wherein the integrated circuitry is electrically coupled to the second electrical contact. In some embodiments, forming the CMUT and the heater disposed in the CMUT includes forming the CMUT on a substrate including integrated circuitry. In some embodiments, the substrate includes a semiconductor chip. In some embodiments, the integrated circuitry includes transmit circuitry, receive circuitry, timing and control circuitry, and/or signal conditioning/processing circuitry.

According to another aspect of the application, a method includes applying a voltage to a heater disposed in a CMUT in an ultrasound imaging device in order to cause the heater to generate heat.

In some embodiments, the CMUT includes a first electrode, a second electrode, and one or more oxide layers disposed on the first electrode, the heater is disposed on the one or more oxide layers and on the second electrode, the heater is electrically coupled to the second electrode, the CMUT is disposed on a substrate including integrated circuitry, and the integrated circuitry is electrically coupled to the second electrode, and applying the voltage to the heater in order to cause the heater to generate heat includes using the integrated circuitry to apply the voltage to the heater through the second electrode. In some embodiments, the integrated circuitry includes transmit circuitry, receive circuitry, timing and control circuitry, and/or signal conditioning/processing circuitry. In some embodiments, the substrate includes a semiconductor chip.

In some embodiments, the CMUT includes a membrane on which the heater is disposed, a first electrical contact disposed on the membrane and electrically coupled to the membrane, and a second electrical contact disposed on the membrane and electrically coupled to the heater, the CMUT is disposed on a substrate including integrated circuitry, and the integrated circuitry is electrically coupled to the second electrical contact, and applying the voltage to the heater in order to cause the heater to generate heat includes using the integrated circuitry is configured to apply a voltage to the heater through the second electrical contact. In some embodiments, the integrated circuitry includes transmit circuitry, receive circuitry, timing and control circuitry, and/or signal conditioning/processing circuitry. In some embodiments, the substrate includes a semiconductor chip.

In some embodiments, the CMUT includes a membrane on which the heater is disposed, a first electrical contact disposed on the membrane and electrically coupled to the membrane, and a second electrical contact disposed on the membrane and electrically coupled to the heater, the ultrasound imaging device includes a DC-DC converter electrically coupled to the second electrical contact, and applying the voltage to the heater in order to cause the heater to generate heat includes using the DC-DC converter to apply the voltage to the heater through the second electrical contact. In some embodiments, applying the voltage to the heater in order to cause the heater to generate heat includes applying a voltage to the heater that causes the heater to heat to a temperature approximately equal to or between 20 to 250 degrees Celsius.

According to another aspect of the application, a method includes receiving, with a processing device in operative communication with an ultrasound imaging device, a first measurement of or relating to a collapse voltage of a CMUT in the ultrasound imaging device at a first time; receiving a second measurement of or relating to the collapse voltage of a CMUT at a second time; determining, based on the first and second measurements, that the collapse voltage of the CMUT has increased by at least a threshold voltage between the first time and the second time; and based on determining that the collapse voltage of the CMUT has increased by at least a threshold voltage between the first time and the second time, automatically causing a voltage to be applied to a heater in the CMUT such that the heater generates heat.

In some embodiments, the first time is after a first ultrasound imaging session and the second time is after a second ultrasound imaging session that is immediately subsequent to the first ultrasound imaging session. In some embodiments, the first time is after a first ultrasound imaging session and the second time is after a second ultrasound imaging session that is a particular number of ultrasound imaging sessions subsequent to the first ultrasound imaging session. In some embodiments, the ultrasound imaging device is configured to perform the first and second measurements, and performing the first and second measurements includes applying a bias voltage to a membrane of the CMUT, inputting a constant current to an electrode of the CMUT such that a voltage ramp is generated across the CMUT, and measuring a time that it takes for the voltage ramp to exceed a reference voltage value, where the first and second measurements are performed at different bias voltages. In some embodiments, receiving the first and second measurements includes receiving measurements of times that it takes for voltage ramps to exceed the reference voltage value at different bias voltages. In some embodiments, receiving the first and second measurements includes receiving measurements of capacitances at different bias voltages. In some embodiments, the method further includes determining the collapse voltage of the CMUT based on detecting a discontinuity in a curve of capacitance versus bias voltage. In some embodiments, automatically causing the voltage to be applied to a heater in the CMUT such that the heater generates heat includes transmitting a command to the ultrasound imaging device to apply the voltage to the heater.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a capacitive micromachined ultrasonic transducer (CMUT) including a heater, in accordance with certain embodiments described herein;

FIG. 2 illustrates a CMUT including a heater, in accordance with certain embodiments described herein;

FIG. 3 illustrates a CMUT including a heater, in accordance with certain embodiments described herein;

FIGS. 4-18 illustrate cross-sections of an ultrasound-on-chip device during its fabrication, where the ultrasound-on-chip device includes the CMUT of FIG. 1, in accordance with certain embodiments described herein;

FIGS. 19-27 illustrate cross-sections of an ultrasound-on-chip device during its fabrication, where the ultrasound-on-chip device includes the CMUT of FIG. 2, in accordance with certain embodiments described herein;

FIGS. 28-33 illustrate cross-sections of an ultrasound-on-chip device during its fabrication, where the ultrasound-on-chip device includes the CMUT of FIG. 3, in accordance with certain embodiments described herein;

FIG. 34 illustrates a top or bottom view of a heater, in accordance with certain embodiments described herein;

FIG. 35 illustrates a top or bottom view of a heater, in accordance with certain embodiments described herein;

FIG. 36 illustrates a top or bottom view of a heater, in accordance with certain embodiments described herein;

FIG. 37 illustrates a process for fabricating a CMUT with a heater disposed in the cavity of the CMUT, in accordance with certain embodiments described herein;

FIG. 38 illustrates a process for fabricating a CMUT with a heater disposed in the CMUT, in accordance with certain embodiments described herein;

FIG. 39 illustrates a process for fabricating a CMUT with a heater disposed in the cavity of the CMUT, in accordance with certain embodiments described herein;

FIG. 40 illustrates a process for fabricating a CMUT with a heater disposed in the CMUT, in accordance with certain embodiments described herein;

FIG. 41 illustrates a process for fabricating a CMUT with a heater disposed in the CMUT, in accordance with certain embodiments described herein;

FIG. 42 illustrates a process for activating a heater in a CMUT based on the collapse voltage of the CMUT, in accordance with certain embodiments described herein;

FIG. 43 illustrates a schematic block diagram of an example ultrasound system, in accordance with certain embodiments described herein;

FIG. 44 illustrates an example handheld ultrasound probe, in accordance with certain embodiments described herein;

FIG. 45 illustrates an example wearable ultrasound patch, in accordance with certain embodiments described herein; and

FIG. 46 illustrates an example ingestible ultrasound pill, in accordance with certain embodiments described herein.

### DETAILED DESCRIPTION

Capacitive micromachined ultrasonic transducers (CMUTs) generally include a cavity, an electrode at the bottom of the cavity, and a membrane at the top of the cavity. When a voltage is applied between the electrode and the membrane, the membrane may vibrate within the cavity, causing transmission of ultrasound signals. Upon reception of ultrasound signals, the membrane may also vibrate and generate changes in voltage between the electrode and the membrane.

A CMUT may operate in four modes: conventional, snap-back, clapping, and collapsed. In conventional mode, the membrane of the CMUT does not contact the bottom of the cavity. In snap-back mode, the CMUT is not in contact with the bottom of the cavity in the biased state, but with the application of an AC pulse, the CMUT membrane touches the bottom of the cavity and snaps back. In clapping mode, when the CMUT is in the biased state, the membrane is in contact with the bottom of the cavity (collapsed), but with the application of an AC pulse to the CMUT, the membrane ceases to be collapsed and then contacts the bottom of the cavity again. In collapse mode, when the CMUT is in the biased state, the membrane is collapsed, and even with the application of an AC pulse to the CMUT, the membrane continues to be in contact with the bottom of the cavity. Clapping and collapse modes may afford a higher transmit pressure as well as higher receive sensitivity. When a CMUT has been operating in clapping and/or collapse modes, it may be desirable for the CMUT membrane to be released from the bottom of the cavity when the CMUT is not operational. However, a CMUT membrane can get stuck on the bottom of the cavity due to electrostatic force from charges trapped in the cavity, or from van der Waals forces between the membrane and the bottom of the cavity. Such stiction is detrimental to the operation of the CMUT. By restricting the motion of the membrane, stiction may cause a lower transmission pressure output as well as decreased reception sensitivity, such that resulting ultrasound images may be lower in quality (e.g., in terms of signal-to-noise ratio (SNR)). Also, in an array of CMUTs, stiction may cause non-uniformity in the array, since some CMUTs might be stuck while others may not. The pattern of stuck CMUTs may also not repeat. Asymmetry in the stiction profile may cause undesirable resonant modes. Non-uniformity and non-repeatability in the stiction profile may particularly negatively affect some imaging modes such as Doppler mode by introducing imaging artifacts.

The inventors have recognized that stiction may be reduced or eliminated by heating. In particular, the inventors have also recognized that a heater disposed in a CMUT may enable heating of the CMUT to reduce or eliminate stiction. The heater may be disposed in the CMUT between the membrane and bottom electrode of the CMUT. The heater may be a planar resistive heater implemented as a thin film layer. The heater may include, for example, a layer of nichrome, chromium-silicon (Cr—Si), or a ferritic iron-chromium-aluminum (FeCrAl) alloy (e.g., Kanthal®). The heater may be formed, for example, using sputtering and/or chemical vapor deposition (CVD). The thickness of the heater may be between or equal to approximately 500-3000 angstroms. In some embodiments, the heater may be disposed at the top of the cavity. For example, the membrane may include a silicon layer and an oxide layer, and the heater may be disposed on the oxide layer adjacent to the cavity. When generating heat, the heater disposed at the top of the cavity may help to relieve stress on the CMUT membrane, which may help the membrane to become unstuck. Also, in the case of a non-evacuated cavity, heating may help to increase the pressure inside the cavity, which may help to release the CMUT membrane when stuck. In some embodiments, the heater may be disposed at the bottom of the cavity. For example, a bottom electrode may have a one or more oxide layers disposed thereon, and the heater may be disposed on one of the oxide layers. As one specific example, a silicon oxide layer may be disposed on the bottom electrode, an aluminum oxide layer may be disposed on the silicon oxide layer, and the heater may be disposed on the aluminum oxide layer, adjacent to the cavity. As another example, a bottom electrode may have two or more oxide layers disposed thereon, and the heater may be disposed between two of the oxide layers. As one specific example, a silicon oxide layer formed using chemical vapor deposition (CVD) may be disposed on the bottom electrode, the heater may be disposed on the silicon oxide layer formed using CVD, and a silicon oxide layer formed using high-density plasma chemical-vapor deposition (HDP-CVD) may be disposed on the heater, adjacent to the cavity. When generating heat, the heater disposed at the bottom of the cavity may help to increase the pressure inside a non-evacuated cavity, which may help to release the CMUT membrane when stuck. Depositing the heater specifically on an oxide surface may be helpful, because after annealing metal alloy thin films (such as the heater), adhesiveness to the oxide surface may be better. Additionally, oxide, which is an insulator, may limit the shorting of the heater to other metals in the CMUT if in contact.

The heater may be laid out, for example, in a shape that includes curved lines, lines at right angles, and/or lines at acute angles. While curved lines may form a circular shape that may allow more uniform distribution of the heater, the deposition of a curved lines may be more challenging than when the heater is laid out in as lines at right angles or lines at acute angles.

In addition to reducing or eliminating stiction, heating may also help to reduce surface, shallow, or deep charges, which may help to lower the charging behavior of the CMUT and help keep the collapse voltage at an acceptably low value. This may help to maintain longevity of the ultrasound imaging device and maintain collection of acceptably high-quality images with the ultrasound imaging device. In some embodiments, the CMUT may include oxide formed with HDP-CVD (abbreviated herein as HDP oxide). HDP oxide may have more oxygen, hydrogen, and/or oxide species present than in oxide formed with regular CVD, and when the HDP oxide is heated, these species may be outgassed. These gasses may increase the pressure in the cavity. As a consequence, when the membrane is pushed down, the gas may help to reduce the impact speed onto the bottom surface of the CMUT. This may help to reduce wear on the bottom surface of the CMUT and increase the longevity of the ultrasound imaging device.

The heater may be capable, for example, of heating to a temperature approximately equal to or between 20 to 250 degrees Celsius. The heater may be activated when the collapse voltage of the CMUT has increased significantly (e.g., >7 V) beyond an initial collapse voltage.

The CMUTs described herein may be integrated on a die (e.g., a semiconductor chip), and thus the heaters described herein, which may be disposed within such CMUTs, may also be integrated on a die. The CMUTs and heaters may be integrated on the same die as ultrasound circuitry, which may include, for example, transmit circuitry (which may include one or more waveform generator and/or pulsers), receive circuitry (which may include one or more receive switches, analog processing circuitry, one or more analog-to-digital converters (ADCs), and/or digital processing circuitry), timing and control circuitry, and/or signal conditioning/processing circuitry (which may include multiplexed digital processing circuitry such as image formation circuitry).

Various aspects of the present disclosure may be used alone, in combination, or in a variety of arrangements, and the disclosure is therefore not limited in its application to the details and arrangement of components set forth in the foregoing description or illustrated in the drawings. For example, aspects described in one embodiment may be combined in any manner with aspects described in other embodiments.

As referred to below, a first structure (e.g., a layer) disposed on a second structure should not be understood to preclude another structure or other structures being disposed between the first and second structures. A first structure (e.g., a layer) formed on a second structure should not be understood to preclude another structure or other structures being formed between the first and second structures. As referred to below, a first structure (e.g., a layer) disposed between a second structure and a third structure should not be understood to preclude another structure or other structures being disposed between the first and second structures or between the first and third structures. A first structure (e.g., a layer) formed between a second structure and a third structure should not be understood to preclude another structure or other structures being formed between the first and second structures or between the first and third structures.

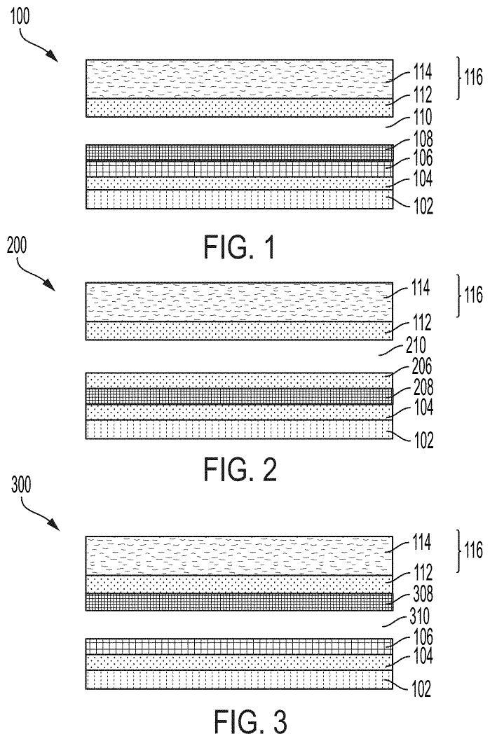

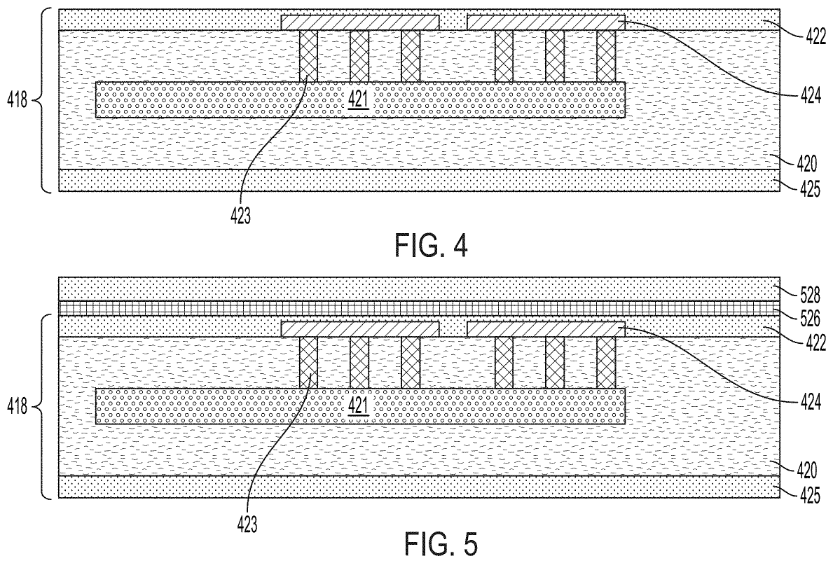

FIG. 1 illustrates a CMUT 100 including a heater 108, in accordance with certain embodiments described herein. The CMUT 100 includes an electrode 102, an oxide (e.g., silicon oxide) layer 104, an oxide (e.g., aluminum oxide) layer 106, the heater 108, a cavity 110, an oxide (e.g., silicon oxide) layer 112, and a silicon layer 114. The oxide layer 112 and the silicon layer 114 constitute the membrane 116 of the CMUT 100. The electrode 102, the oxide layer 104, the oxide layer 106, and the heater 108 are at the bottom of the cavity 110 and the membrane 116 is at the top of the cavity 110. The oxide layer 104 is disposed on the electrode 102, the oxide layer 106 is disposed on the oxide layer 104, the heater 108 is disposed on the oxide layer 106, and the heater 108 is adjacent to the cavity 110. In some embodiments, one of the oxide layer 104 or the oxide layer 106 may be absent, and the heater 108 may be disposed on the oxide layer not absent. The oxide layer 112 is disposed on the silicon layer 114 and is adjacent to the cavity 110. When generating heat, the heater 108 disposed at the bottom of the cavity 110 may help to increase the pressure inside the cavity 110 when non-evacuated, which may help to release the membrane 116 when stuck. Further description of forming the CMUT 100 may be found with reference to FIGS. 4-18.

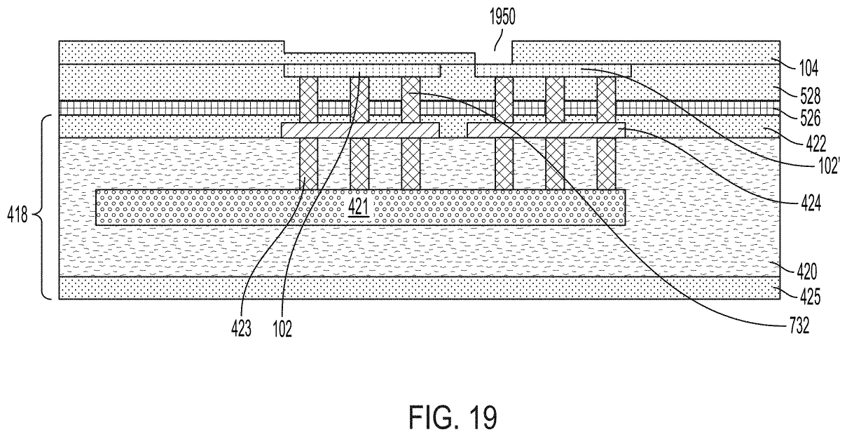

FIG. 2 illustrates a CMUT 200 including a heater 208, in accordance with certain embodiments described herein. The CMUT 200 includes the electrode 102, the oxide layer 104, the heater 208, an oxide layer 206, a cavity 210, the oxide layer 112, and the silicon layer 114. The oxide layer 112 and the silicon layer 114 constitute the membrane 116 of the CMUT 200. The electrode 102, the oxide layer (e.g., an oxide layer deposited with CVD) 104, the heater 208, and the oxide layer (e.g., an oxide layer deposited with HDP-CVD) 206 are at the bottom of the cavity 210 and the membrane 116 is at the top of the cavity 210. The oxide layer 104 is disposed on the electrode 102, the heater 208 is disposed on the oxide layer 104, the oxide layer 206 is disposed on the heater 208, and the oxide layer 206 is adjacent to the cavity 210. The oxide layer 112 is disposed on the silicon layer 114, and the oxide layer 112 and is adjacent to the cavity 210. When generating heat, the heater 208 disposed at the bottom of the cavity 210 may help to increase the pressure inside the cavity 210 when non-evacuated, which may help to release the membrane 116 when stuck. Embedding the heater 208, rather than having a heater adjacent to cavity (e.g., in the case of the heaters 108 and 308) may enable a smaller size for the cavity 210, which may help to keep the collapse voltage at an acceptably low value. Embedding the heater 208 may also enable the thickness of the heater 208 to be larger. Increasing the thickness of the heater 208 may be helpful in increasing resistance of the heater 208 to moisture and decreasing the inductance and capacitance of the heater 208. Further description of the CMUT 200 may be found with reference to FIGS. 19-27.

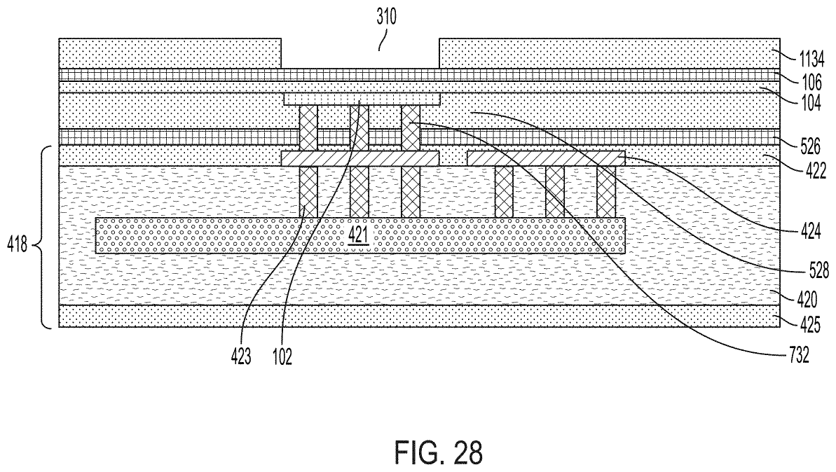

FIG. 3 illustrates a CMUT 300 including a heater 308, in accordance with certain embodiments described herein. The CMUT 300 includes the electrode 102, the oxide layer 104, the oxide layer 106, the heater 308, a cavity 310, the oxide layer 112, and the silicon layer 114. The oxide layer 112 and the silicon layer 114 constitute the membrane 116 of the CMUT 300, and the heater 308 is disposed on the membrane 116. The electrode 102, the oxide layer 104, and the oxide layer 106 are at the bottom of the cavity 310 and the membrane 116 and the heater 308 are at the top of the cavity 310. The oxide layer 104 is disposed on the electrode 102, the oxide layer 106 is disposed on the oxide layer 104, and the oxide layer 106 is adjacent to the cavity 310. In some embodiments, one of the oxide layer 104 or the oxide layer 106 may be absent. The oxide layer 112 is disposed on the silicon layer 114, the heater 308 is disposed on the oxide layer 112, and the heater 308 is adjacent to the cavity 310. When generating heat, the heater 308 disposed at the top of the cavity 310 may help to relieve stress on the membrane 116, which may help the membrane 116 to become unstuck. Also, heating may help increase the pressure inside the cavity 310 when non-evacuated, which may help to release the membrane 116 when stuck. Further description of the CMUT 300 may be found with reference to FIGS. 28-33.

FIGS. 4-18 illustrate cross-sections of an ultrasound-on-chip device during its fabrication, where the ultrasound-on-chip device includes the CMUT 100, in accordance with certain embodiments described herein. The ultrasound-on-chip device may be disposed in an ultrasound imaging device (e.g., the ultrasound imaging device 4304). As referred to herein, an ultrasound-on-chip device may include ultrasonic transducers and ultrasound circuitry integrated onto one or more semiconductor dies. Ultrasound circuitry may refer to circuitry involved in driving ultrasonic transducers to transmit ultrasound waves and circuitry involved in receiving and processing ultrasound waves. Further aspects of such ultrasound-on-chip devices are described in U.S. patent application Ser. No. 15/415,434 titled “UNIVERSAL ULTRASOUND DEVICE AND RELATED APPARATUS AND METHODS,” filed on Jan. 25, 2017 and published as U.S. Pat. Publication No. 2017/0360397 A1 (and assigned to the assignee of the instant application), which is incorporated by reference herein in its entirety.

As illustrated in FIG. 4, a substrate 418 includes a base layer (e.g., a bulk silicon wafer) 420, an insulating layer 422, metallization 424, integrated circuitry 421, and vias 423. An insulating layer 425 is formed on the backside of the base layer 420. The substrate 418 may be a die and may be a semiconductor chip (e.g., a complementary metal oxide semiconductor (CMOS) substrate) fabricated at a commercial foundry. The integrated circuitry 421 may include semiconductor structures such as transistors as part of front-end-of-line (FEOL) processes and is electrically coupled through the vias 423 to the metallization 424. The integrated circuitry 421 may include, for example, transmit circuitry (which may include one or more waveform generator and/or pulsers), receive circuitry (which may include one or more receive switches, analog processing circuitry, one or more analog-to-digital converters (ADCs), and/or digital processing circuitry), timing and control circuitry, and/or signal conditioning/processing circuitry (which may include multiplexed digital processing circuitry such as image formation circuitry). The metallization 424 may be formed as part of back-end-of-line (BEOL) processes. The metallization 424 may be formed of aluminum, copper, or any other suitable metallization material. As one example, the metallization 424 may serve as routing layers or other functions. In some embodiments, the metallization 424 may be electrically connected to other metallization (e.g., routing layers) within the base layer 420. In some embodiments, the metallization 424 may be a redistribution layer (which may be post-processed, and may be made of an aluminum-copper alloy) that is electrically connected to other metallization within the base layer 420. Thus, in practice, the substrate 418 may include more than one metallization layer and/or redistribution layer (which may be post-processed), but for simplicity only one metallization 424 is illustrated.

As illustrated in FIG. 5, a nitride layer 526 and an oxide layer 528 are formed on the substrate 418. The nitride layer 526 may be formed, for example, by plasma enhanced chemical vapor deposition (PECVD). The oxide layer 528 may be formed, for example, by PECVD of oxide.

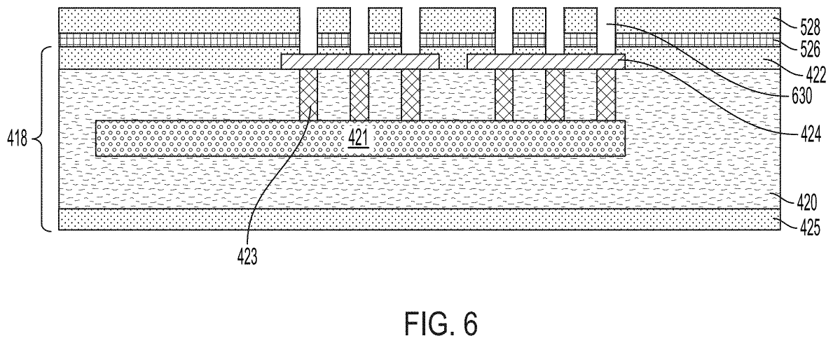

As illustrated in FIG. 6, openings 630 are formed down from the oxide layer 528 to the metallization 424. Such openings may be formed, for example, with lithography.

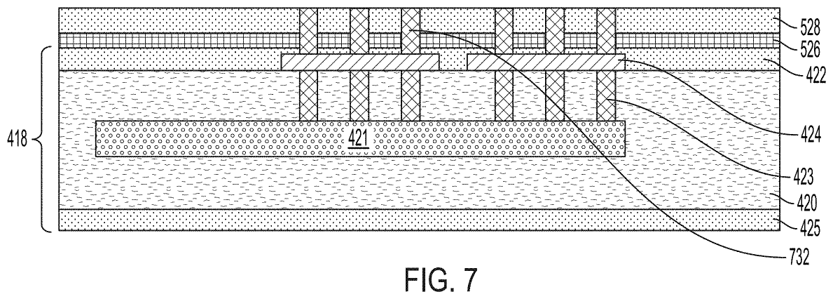

As illustrated in FIG. 7, vias 732 are formed in the openings 630. The vias 732 may include, for example, tungsten or copper, and may be formed by deposition followed by chemical mechanical polishing (CMP).

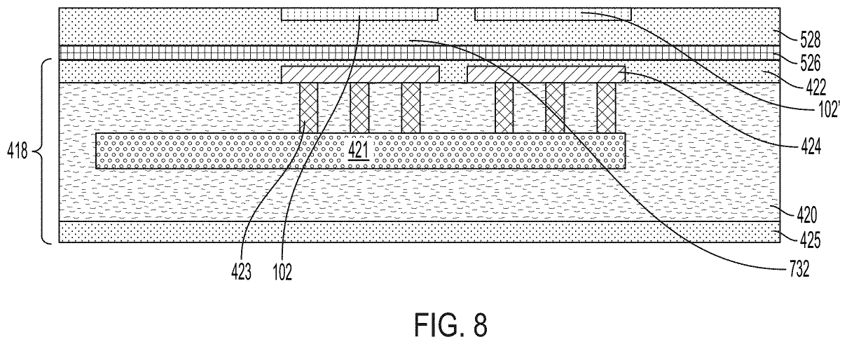

As illustrated in FIG. 8, electrodes 102 and 102′ are formed on the vias 732 and further oxide is formed on the oxide layer 528 around the electrodes 102 and 102′. The electrodes 102 and 102′ may include, for example, titanium, titanium nitride, tungsten, and/or copper. In embodiments in which the electrodes 102 and 102′ and the vias 732 include tungsten, they may be formed with a dual damascene process. In some embodiments, the electrodes 102 and 102′ may be formed from a sea (i.e., a large plurality) of tungsten vias formed with a single damascene process. In some embodiments, the electrodes 102 and 102′ and the vias 732 may include copper. In some embodiments, the electrodes 102 and 102′ may be formed by depositing a layer of material for the electrodes 102 and 102′ and a CMP stop layer (e.g., nitride) on top of the electrode layer. The electrode layer may be patterned using lithography to form the individual electrodes 102 and 102′. Further oxide may be formed on top of the oxide layer 528 (e.g., using HDP-CVD) and CMP may then be used to remove the oxide down to the CMP stop layer, which may then be etched away. While two electrodes 102 and 102′ are shown, it should be appreciated that more than two electrodes may be formed in the ultrasound-on-chip device.

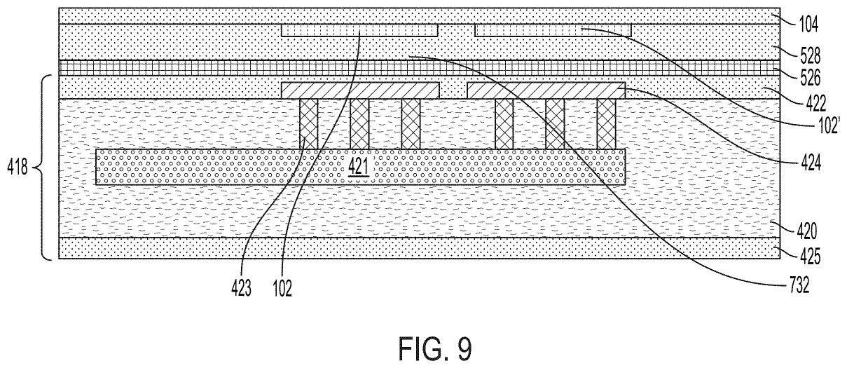

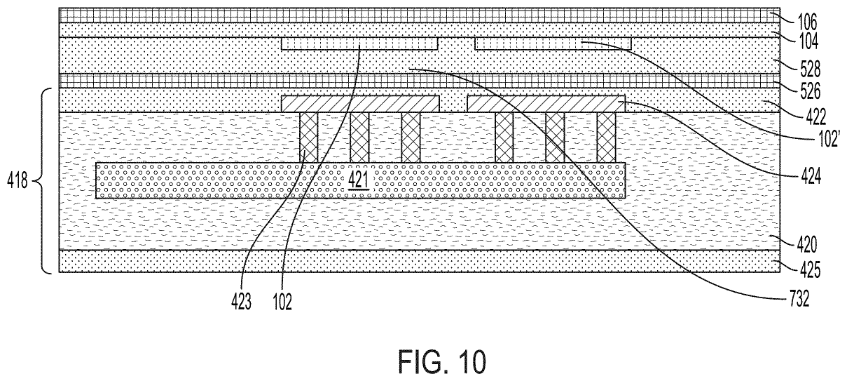

As illustrated in FIG. 9, the oxide layer 104 is formed on the electrodes 102 and 102′ and the oxide layer 528. In some embodiments, the oxide layer 104 may be silicon oxide. In some embodiments, the oxide layer 104 may be formed using CVD. In some embodiments, the oxide layer 104 may be formed using HDP-CVD.

As illustrated in FIG. 10, the oxide layer 106 is formed on the oxide layer 106. In some embodiments, the oxide layer 106 may be aluminum oxide. In some embodiments, the oxide layer 106 may be formed using atomic layer deposition (ALD).

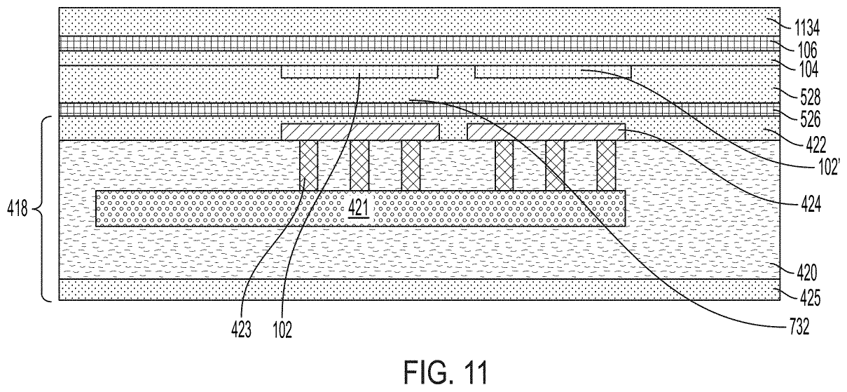

As illustrated in FIG. 11, an oxide layer 1134 is formed on the oxide layer 106. In some embodiments, the oxide layer 1134 may be formed with HDP-CVD followed by CMP to reduce surface roughness for subsequent fusion bonding to the oxide layer 1134.

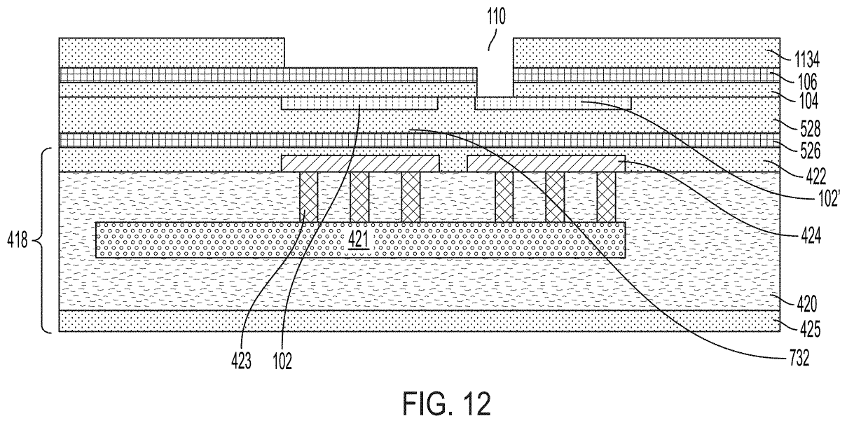

As illustrated in FIG. 12, the cavity 110 is formed. The cavity 110 extends down through the oxide layer 1134 to the oxide layer 106 above the electrode 102 and down through the oxide layer 1134, the oxide layer 106, and the oxide layer 104 to the electrode 102′. In some embodiments, the cavity 110 may be formed using lithography. While one cavity 110 is shown in FIG. 12, it should be appreciated that more than one cavity 1100 may be formed in the ultrasound-on-chip device.

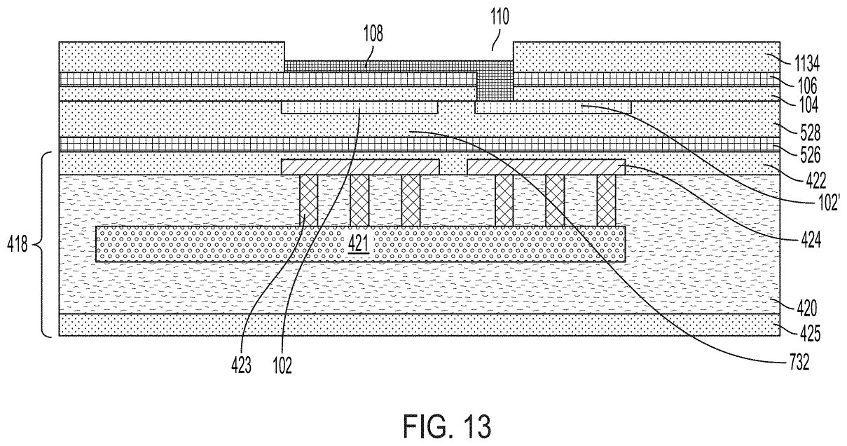

As illustrated in FIG. 13, the heater 108 is formed in the cavity 110. The heater 108 may be a planar resistive heater including, for example, a thin film layer of nichrome, chromium-silicon (Cr—Si), or a ferritic iron-chromium-aluminum (FeCrAl) alloy (e.g., Kanthal®). The heater 108 may be formed, for example, using sputtering and/or chemical vapor deposition (CVD). The thickness of the heater 108 may be between or equal to approximately 500-1500 angstroms. The heater 108 is formed on top of the oxide layer 106 above the electrode 102 and also extends down to the electrode 102′, such that the electrode 102′ is electrically coupled to the heater 108. The heater 108 may be laid out, for example, in a shape that includes curved lines, lines at right angles, and/or lines at acute angles. While curved lines may form a circular shape that may allow more uniform distribution of the heater 108, the deposition of a curved lines may be more challenging than when the heater 108 is laid out in as lines at right angles or lines at acute angles. Depositing the heater 108 specifically on an oxide surface, namely the oxide layer 106, may be helpful, because after annealing the heater 108, adhesiveness of the heater 108 to the oxide layer 106 may be better. Additionally, the oxide layer 106, which is an insulator, may limit the shorting of the heater 108 to other metals in the ultrasound-on-chip device. The heater 108 may be capable, for example, of heating to a temperature approximately equal to or between 20 to 250 degrees Celsius.

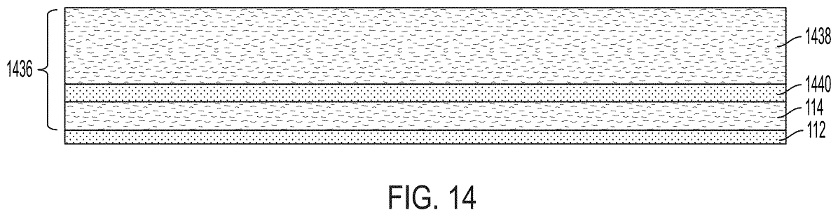

As illustrated in FIG. 14, a substrate 1436 (which will provide the membrane 116 to seal the cavity 110) is illustrated. The substrate 1436 may include, for example, a silicon-on-insulator (SOI) substrate that includes a handle (e.g., silicon) layer 1438, an oxide layer 1440 (which may be a buried oxide (BOX) layer) of the SOI substrate), and the silicon layer 114 (which may be a silicon device layer of the SOI substrate). The silicon layer 114 may be formed of single crystal silicon and may be doped in some embodiments. In some embodiments, the silicon layer 114 may be highly doped P-type, although N-type doping may alternatively be used. When doping is used, the doping may be uniform or may be patterned (e.g., by implanting in patterned regions). The silicon layer 114 may already be doped when the SOI wafer is procured, or may be doped by ion implantation, as the manner of doping is not limiting. In some embodiments, the silicon layer 114 may be formed of polysilicon or amorphous silicon. In some embodiments, the silicon layer 114 may be undoped. The oxide layer 112 is formed on the silicon layer 114. The oxide layer 112 may be a thermal oxide, but it should be appreciated that oxides other than thermal oxide may alternatively be used.

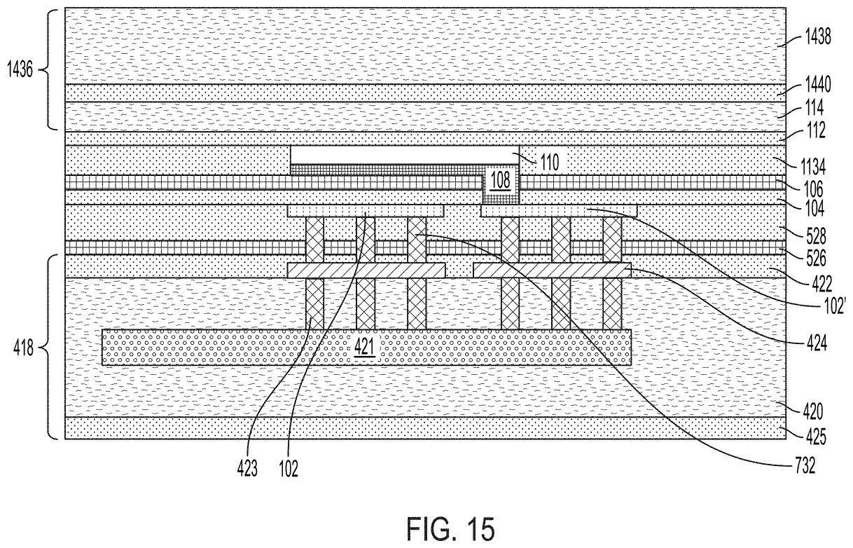

As illustrated in FIG. 15, the oxide layer 1134 on the substrate 418 and the oxide layer 112 on the substrate 1436 are bonded together. Thus, in the example of FIG. 15, the bond is an oxide-oxide bond. In some embodiments, the bond may be a fusion bond. The bonding seals the cavity 110. Such bonding may, in some embodiments, involve only the use of low temperature (e.g., below 450° C.) which may prevent damage to the integrated circuitry 421 in the substrate 418.

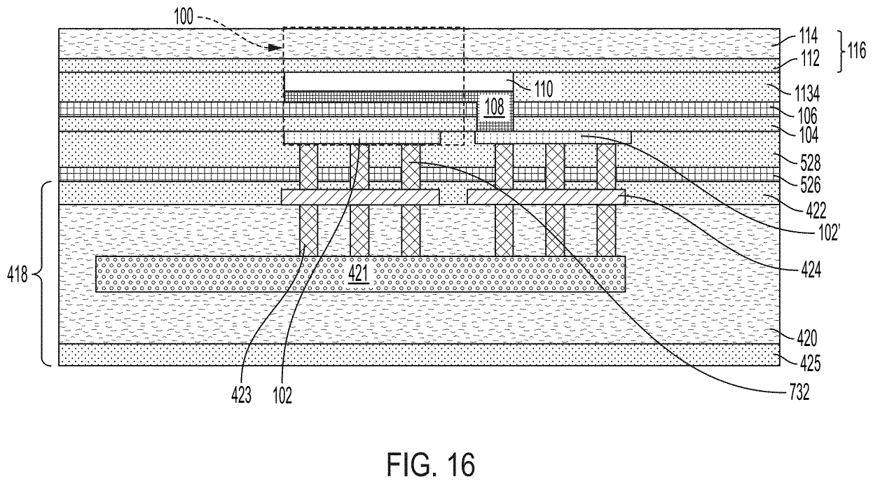

As illustrated in FIG. 16, the oxide layer 1440 and the handle layer 1438 of the substrate 1436 are removed. For example, grinding, etching, or any other suitable technique or combination of techniques may be used. FIG. 16 further illustrates that the CMUT 100 has been formed.

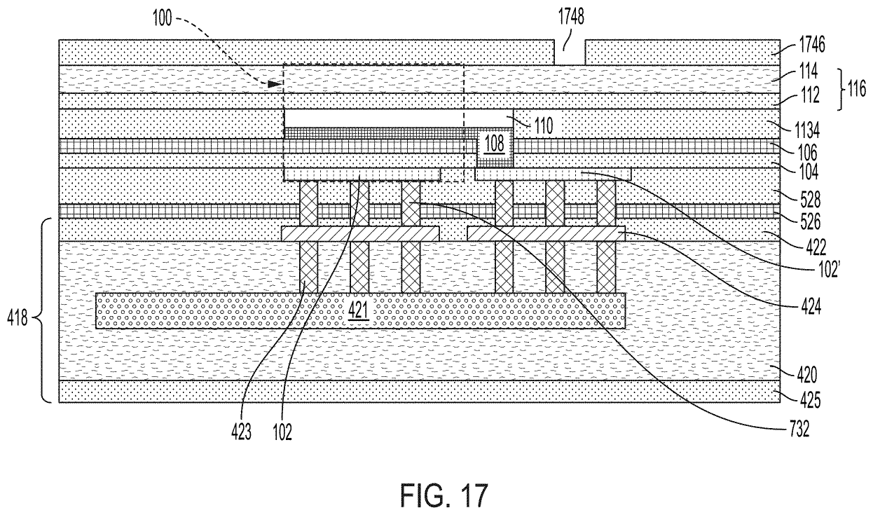

As illustrated in FIG. 17, an oxide layer 1746 is formed on the silicon layer 114. An opening 1748 is formed in the oxide layer 1746 (e.g., using lithography).

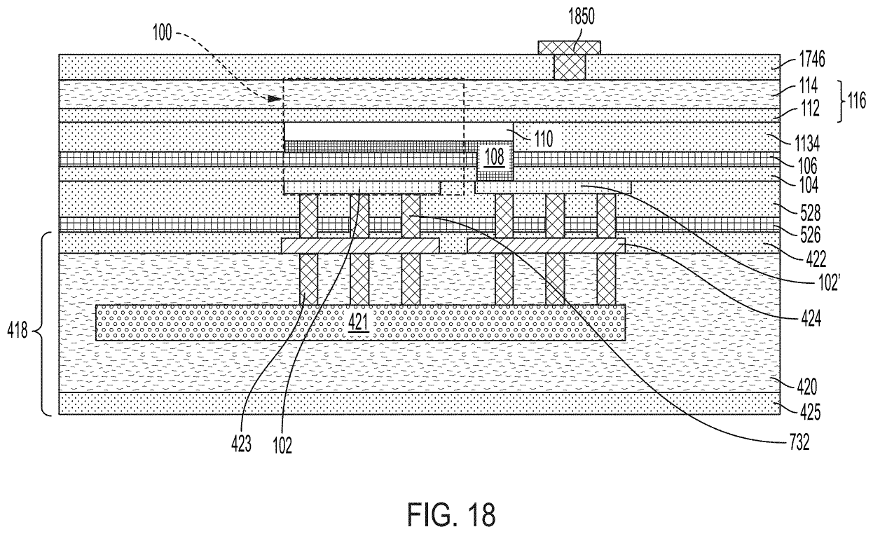

As illustrated in FIG. 18, a contact 1850 is formed in the opening 1748 and on the oxide layer 1746. In some embodiments, the contact 1850 may include a stack of titanium, aluminum-copper alloy, titanium nitride, aluminum-copper alloy, and titanium nitride. The contact 1850 extends to the silicon layer 114 of the membrane 116, such that the contact 1850 is electrically coupled to the silicon layer 114 of the membrane 116. It should be appreciated that further processing and packaging of the ultrasound-on-chip device may occur.

In some embodiments, circuitry external to the ultrasound-on-chip device but internal to the ultrasound imaging device (e.g., a DC-DC converter such as a charge pump, not illustrated) may apply a voltage to the membrane 116 through the contact 1850. In some embodiments, the integrated circuitry 421 in the substrate 418 may apply a voltage to the electrode 102 through the vias 423, the metallization 424, and the vias 732. A voltage may thereby be established between the membrane 116 and the electrode 102 of the CMUT 100. In some embodiments, the integrated circuitry 421 in the substrate 418 may also apply a voltage to the heater 108 through the vias 423, the metallization 424, the vias 732, and the electrode 102′. Applying a voltage to the heater 108 may cause the heater 108 to heat. The relationship between voltage applied to the heater 108 and temperature of the heater 108 may be approximately linear.

It should be appreciated that the CMUT 100 and the heater 108 disposed in the CMUT 100 are integrated on the same substrate 418 (which may be, for example, a semiconductor chip) as the integrated circuitry 421 (which may include, for example, transmit circuitry, receive circuitry, control circuitry, power management circuitry, and/or processing circuitry).

FIGS. 19-27 illustrate cross-sections of an ultrasound-on-chip device during its fabrication, where the ultrasound-on-chip device includes the CMUT 200, in accordance with certain embodiments described herein. The ultrasound-on-chip device may be disposed in an ultrasound imaging device (e.g., the ultrasound imaging device 4302).

The fabrication proceeds as described with reference to FIGS. 4-9. As illustrated in FIG. 19, an opening 1950 is formed in the oxide layer 104 (e.g., using lithography). The opening 1950 extends partially through the oxide layer 104 above the electrode 102 and through the oxide layer 104 down to the electrode 102′.

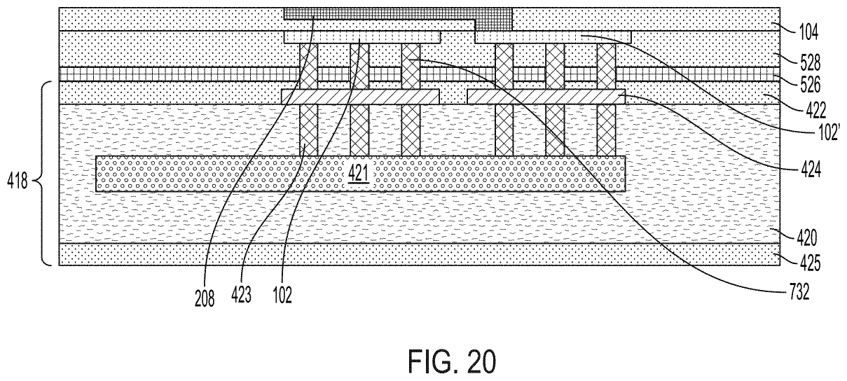

As illustrated in FIG. 20, the heater 208 is formed in the opening 1950. The heater 208 may be a planar resistive heater including, for example, a layer of nichrome, chromium-silicon (Cr—Si), or a ferritic iron-chromium-aluminum (FeCrAl) alloy (e.g., Kanthal®). The heater 208 may be formed, for example, using sputtering and/or chemical vapor deposition (CVD). The thickness of the heater 208 may be between or equal to approximately 500-3000 angstroms. The heater 208 is formed on top of the oxide layer 104 above the electrode 102 and also extends down to the electrode 102′, such that the heater 208 is electrically coupled to the electrode 102′. The heater 208 may be laid out, for example, in a shape that includes curved lines, lines at right angles, and/or lines at acute angles. While curved lines may form a circular shape that may allow more uniform distribution of the heater 208, the deposition of a curved lines may be more challenging than when the heater 208 is laid out in as lines at right angles or lines at acute angles. Depositing the heater 208 specifically on an oxide surface, namely the oxide layer 104, may be helpful, because after annealing the heater 208, adhesiveness of the heater 208 to the oxide layer 104 may be better. Additionally, the oxide layer 104, which is an insulator, may limit the shorting of the heater 208 to other metals in the ultrasound-on-chip device. The heater 208 may be capable, for example, of heating to a temperature approximately equal to or between 20 to 250 degrees Celsius.

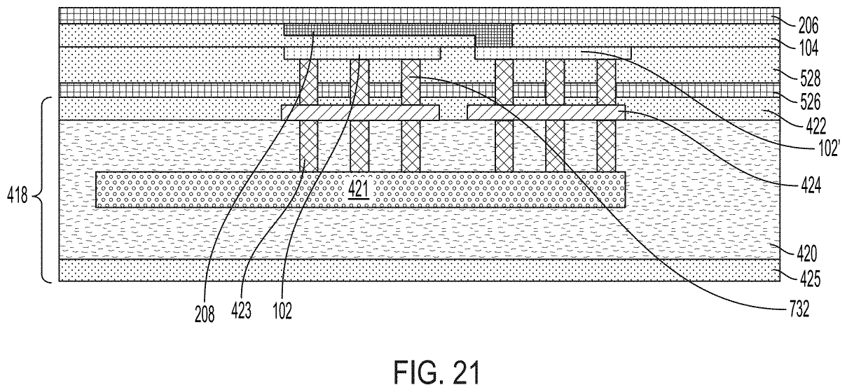

As illustrated in FIG. 21, the oxide (e.g., silicon oxide) layer 206 is formed on the heater 208 and the oxide layer 104. The heater 208 is thus embedded between two oxide layers, the oxide layer 104 and the oxide layer 206. The oxide layer 206 may be HDP oxide. When the oxide layer 104 is CVD oxide, the heater 208 may be embedded between a CVD oxide layer and an HDP oxide layer. When the oxide layer 104 is HDP oxide, the heater 208 may be embedded between two HDP oxide layers.

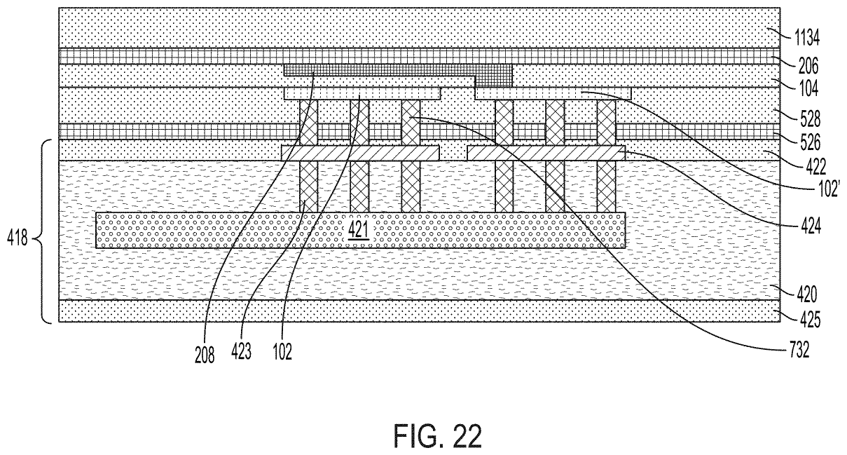

As illustrated in FIG. 22, the oxide layer 1134 is formed on the oxide layer 206. In some embodiments, the oxide layer 1134 may be formed with HDP-CVD followed by CMP to reduce surface roughness for subsequent fusion bonding to the oxide layer 1134.

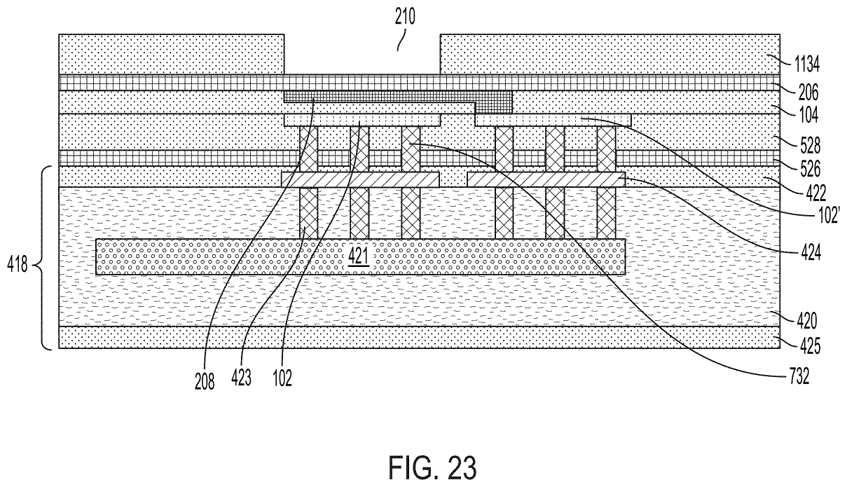

As illustrated in FIG. 23, the cavity 210 is formed. The cavity 210 extends down through the oxide layer 1134 to the oxide layer 206 above the electrode 102. In some embodiments, the cavity 210 may be formed using lithography. While one cavity 210 is shown in FIG. 23, it should be appreciated that more than one cavity 210 may be formed in the ultrasound-on-chip device.

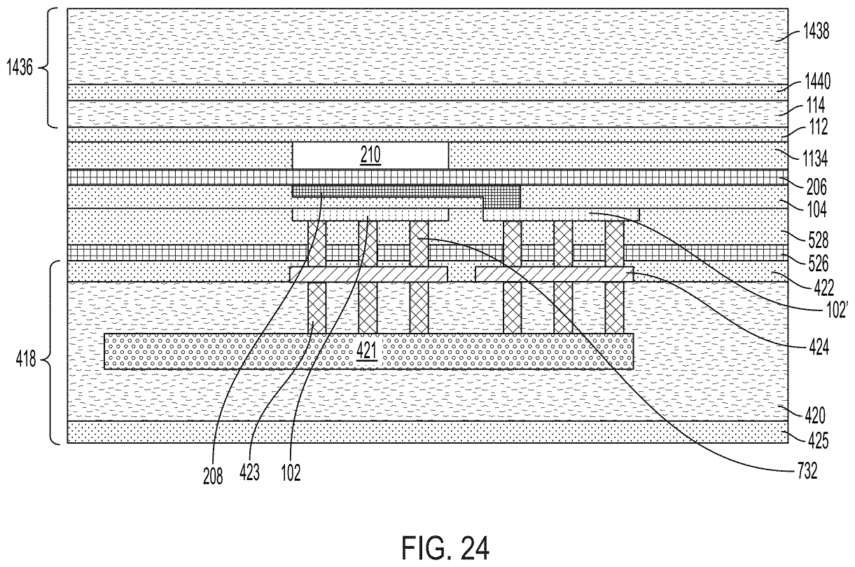

As illustrated in FIG. 24, the oxide layer 1134 of the substrate 418 and the oxide layer 112 of the substrate 1436 are bonded together. Thus, in the example of FIG. 24, the bond is an oxide-oxide bond. In some embodiments, the bond may be a fusion bond. The bonding seals the cavity 210. Such bonding may, in some embodiments, involve only the use of low temperature (e.g., below 450° C.) which may prevent damage to the integrated circuitry 421 in the substrate 418.

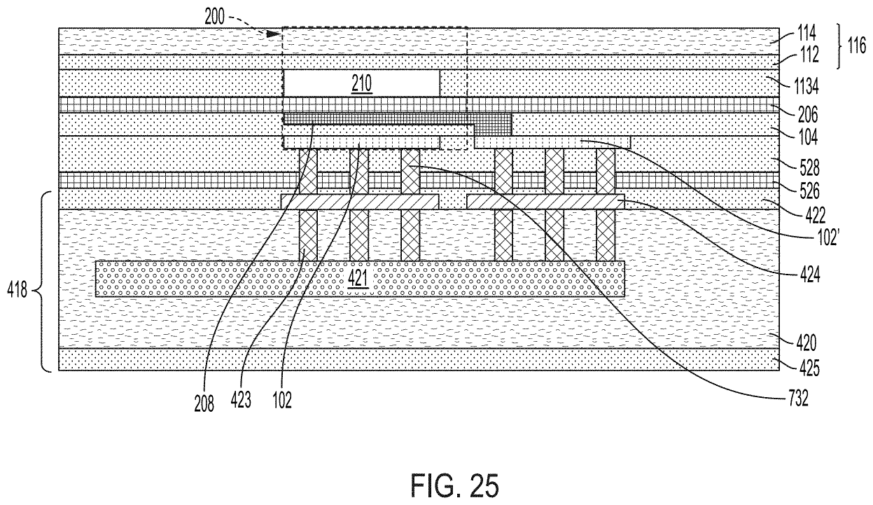

As illustrated in FIG. 25, the oxide layer 1440 and the handle layer 1438 of the substrate 1436 are removed. For example, grinding, etching, or any other suitable technique or combination of techniques may be used. FIG. 25 further illustrates that the CMUT 200 has been formed.

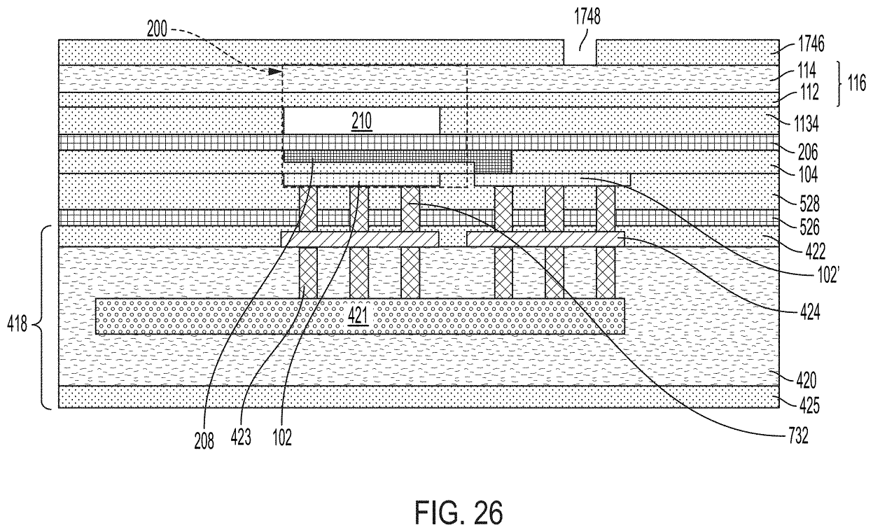

As illustrated in FIG. 26, the oxide layer 1746 is formed on the silicon layer 114. The opening 1748 is formed in the oxide layer 1746 (e.g., using lithography).

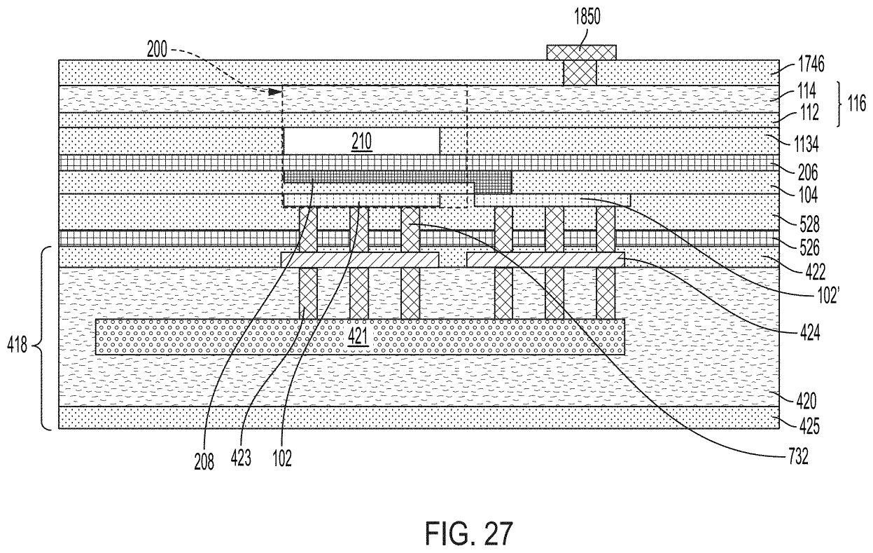

As illustrated in FIG. 27, the contact 1850 is formed in the opening 1748 and on the oxide layer 1746, such that the contact 1850 is electrically coupled to the silicon layer 114 of the membrane 116. In some embodiments, the contact 1850 may include a stack of titanium, aluminum-copper alloy, titanium nitride, aluminum-copper alloy, and titanium nitride. It should be appreciated that further processing and packaging of the ultrasound-on-chip device may occur.

In some embodiments, circuitry external to the ultrasound-on-chip device but internal to the ultrasound imaging device (e.g., a DC-DC converter such as a charge pump, not illustrated) may apply a voltage to the membrane 116 through the contact 1850. In some embodiments, the integrated circuitry 421 in the substrate 418 may apply a voltage to the electrode 102 through the vias 423, the metallization 424, and the vias 732. A voltage may thereby be established between the membrane 116 and the electrode 102 of the CMUT 200. In some embodiments, the integrated circuitry 421 in the substrate 418 may also apply a voltage to the heater 208 through the vias 423, the metallization 424, the vias 732, and the electrode 102′. Applying a voltage to the heater 208 may cause the heater 208 to heat. The relationship between voltage applied to the heater 208 and temperature of the heater 208 may be approximately linear.

It should be appreciated that the CMUT 200 and the heater 208 disposed in the CMUT 200 are integrated on the same substrate 418 (which may be, for example, a semiconductor chip) as the integrated circuitry 421 (which may include, for example, transmit circuitry, receive circuitry, control circuitry, power management circuitry, and/or processing circuitry).

FIGS. 28-33 illustrate cross-sections of an ultrasound-on-chip device during its fabrication, where the ultrasound-on-chip device includes the CMUT 300, in accordance with certain embodiments described herein. The ultrasound-on-chip device may be disposed in an ultrasound imaging device (e.g., the ultrasound imaging device 4302).

The fabrication proceeds as described with reference to FIGS. 4-11. For simplicity, the electrode 102′ is not illustrated. As illustrated in FIG. 28, the cavity 310 is formed. The cavity 210 extends down through the oxide layer 1134 to the oxide layer 106 above the electrode 102. In some embodiments, the cavity 310 may be formed using lithography. While one cavity 310 is shown in FIG. 28, it should be appreciated that more than one cavity 310 may be formed in the ultrasound-on-chip device.

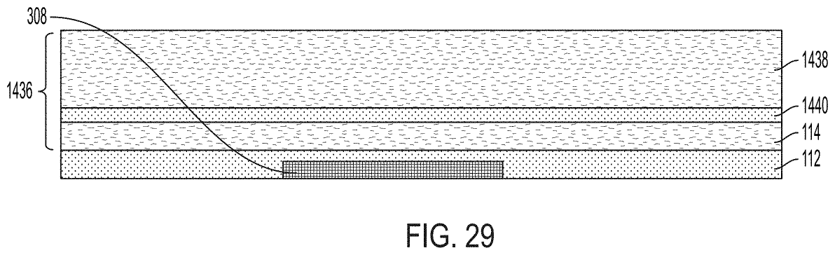

FIG. 29 illustrates the substrate 1436. The heater 308 is formed on the oxide layer 112. Further oxide is formed around the heater 308 on the oxide layer 112. The heater 308 may be a planar resistive heater including, for example, a layer of nichrome, chromium-silicon (Cr—Si), or a ferritic iron-chromium-aluminum (FeCrAl) alloy (e.g., Kanthal®). The heater 308 may be formed, for example, using sputtering and/or chemical vapor deposition (CVD). The thickness of the heater 308 may be between or equal to approximately 500-1500 angstroms. The heater 308 may be laid out, for example, in a shape that includes curved lines, lines at right angles, and/or lines at acute angles. While curved lines may form a circular shape that may allow more uniform distribution of the heater 308, the deposition of a curved lines may be more challenging than when the heater 308 is laid out in as lines at right angles or lines at acute angles. Depositing the heater 308 specifically on an oxide surface, namely the oxide layer 112, may be helpful, because after annealing the heater 308, adhesiveness of the heater 308 to the oxide layer 112 may be better. Additionally, the oxide layer 112, which is an insulator, may limit the shorting of the heater 308 to other metals in the CMUT 300 if in contact. The heater 308 may be capable, for example, of heating to a temperature approximately equal to or between 20 to 250 degrees Celsius.

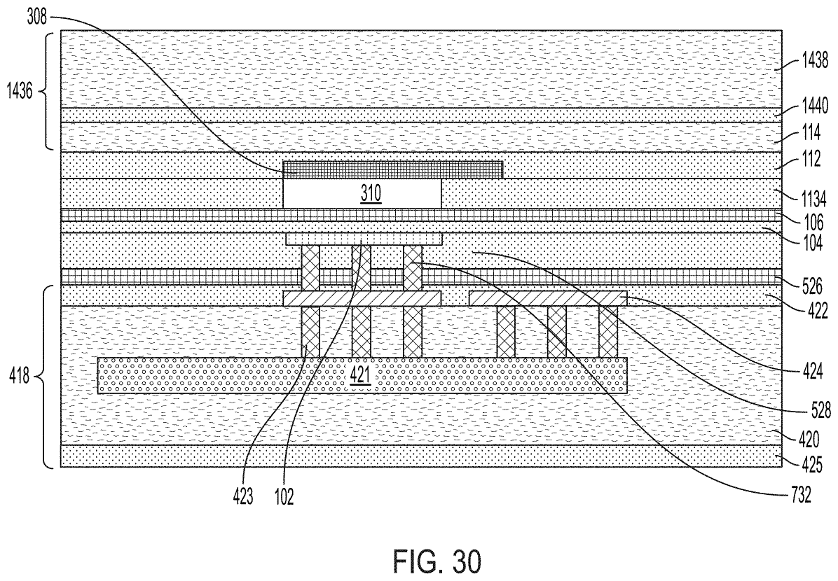

As illustrated in FIG. 30, the oxide layer 1134 of the substrate 418 and the oxide layer 112 of the substrate 1436 are bonded together. In the example of FIG. 30, the bond is an oxide-oxide bond. In some embodiments, the bond may be a fusion bond. The bonding seals the cavity 210. Such bonding may, in some embodiments, involve only the use of low temperature (e.g., below 450° C.) which may prevent damage to circuitry on the substrate 418. In some embodiments, an aligned bond may be performed, such that the heater 208 is aligned with the cavity 210 upon bonding.

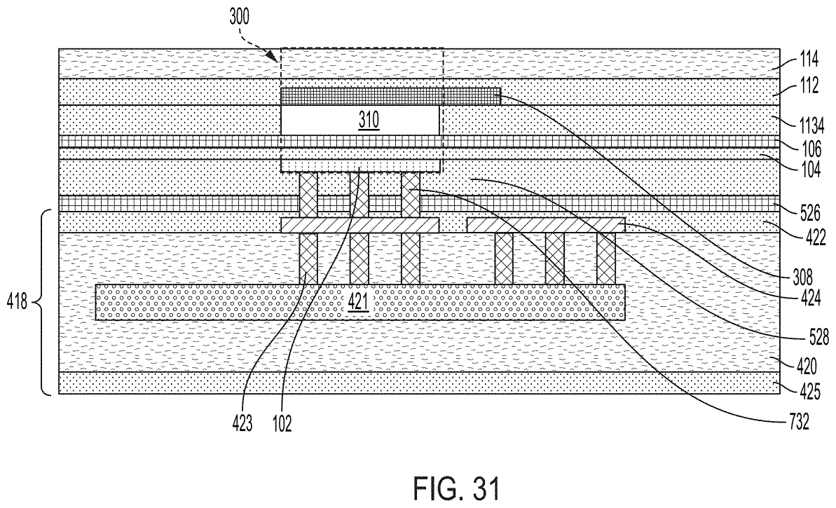

As illustrated in FIG. 31, the oxide layer 1440 and the handle layer 1438 of the substrate 1436 are removed. For example, grinding, etching, or any other suitable technique or combination of techniques may be used. FIG. 31 further illustrates that the CMUT 300 has been formed.

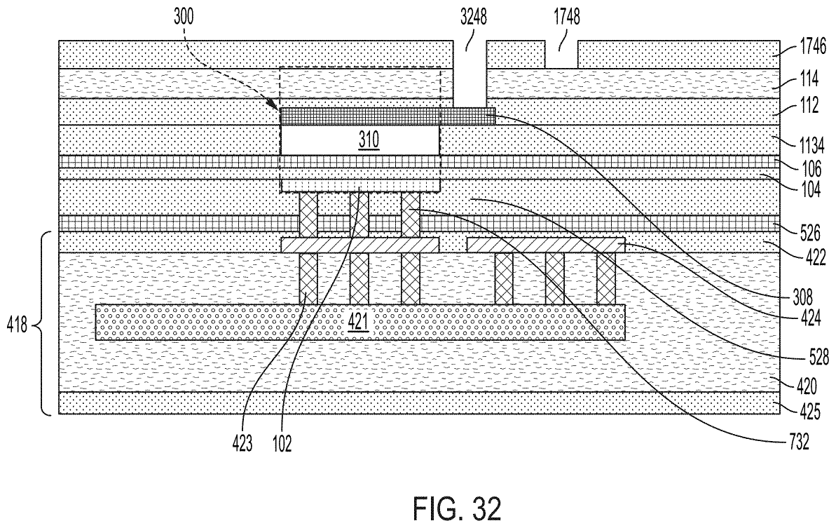

As illustrated in FIG. 32, the oxide layer 1746 is formed on the silicon layer 114. The opening 1748 is formed in the oxide layer 1746 (e.g., using lithography). An opening 3248 is formed extending through the oxide layer 1746, the silicon layer 114, and the oxide layer 112 down to the heater 308.

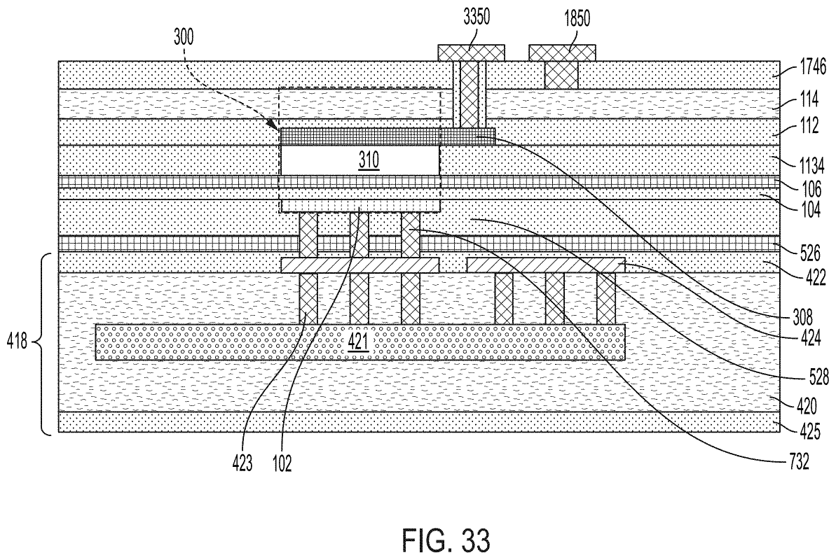

As illustrated in FIG. 33, the contact 1850 is formed in the opening 1748 and on the oxide layer 1746, such that the contact 1850 is electrically coupled to the silicon layer 112 of the membrane 116. A contact 3350 is formed in the opening 3248, such that the contact 3350 is electrically coupled to the heater 308. The contact 3350 is electrically isolated from the silicon layer 114 of the membrane 116 (e.g., by oxide). In some embodiments, the contacts 1850 and 3350 may each include a stack of titanium, aluminum-copper alloy, titanium nitride, aluminum-copper alloy, and titanium nitride. It should be appreciated that further processing and packaging of the ultrasound-on-chip device may occur.

In some embodiments, circuitry external to the ultrasound-on-chip device but internal to the ultrasound imaging device (e.g., a DC-DC converter such as a charge pump, not illustrated) may apply a voltage to the membrane 116 through the contact 1850. In some embodiments, the integrated circuitry 421 in the substrate 418 may apply a voltage to the electrode 102 through the vias 423, the metallization 424, and the vias 732. A voltage may thereby be established between the membrane 116 and the electrode 102 of the CMUT 200. In some embodiments, circuitry external to the ultrasound-on-chip device but internal to the ultrasound imaging device (e.g., the DC-DC converter such as the charge pump) may also apply a voltage to the heater 308 through the contact 3350. Applying a voltage to the heater 308 may cause the heater 308 to heat. In some embodiments, the integrated circuitry 421 may apply a voltage to the heater 308 through the contact 3350. For example, the integrated circuitry 421 may be electrically coupled through vias and/or electrical contacts in the ultrasound-on-chip and/or packaging to the contact 3350 (not illustrated). The relationship between voltage applied to the heater 308 and temperature of the heater 308 may be approximately linear.

It should be appreciated that the CMUT 300 and the heater 308 disposed in the CMUT 300 are integrated on the same substrate 418 (which may be, for example, a semiconductor chip) as the integrated circuitry 421 (which may include, for example, transmit circuitry, receive circuitry, control circuitry, power management circuitry, and/or processing circuitry).

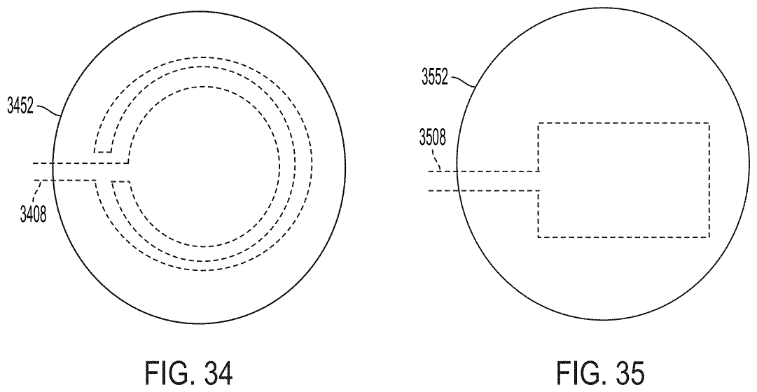

FIG. 34 illustrates a top or bottom view of a heater 3408 (which may be the same as the heaters 108, 208, or 308) in a CMUT (which may be the same as the CMUT 100, 200, or 300), in accordance with certain embodiments described herein. The heater 3408 is disposed on a surface 3452. The surface 3452 may be at the bottom of the cavity in a CMUT or at the top of the cavity in a CMUT. For example, the surface 3452 may be above an electrode (e.g., the electrode 102) or a CMUT or on a membrane (e.g., the membrane 116) of a CMUT. In embodiments in which the surface 3452 is above the electrode, the surface may be a top surface of the oxide layer 106 or the oxide layer 104, and the view in FIG. 34 may be a top view. In embodiments in which the surface 3452 is on a membrane, the surface may be a bottom surface of the oxide layer 112 and the view in FIG. 34 may be a bottom view. The heater 3408 includes curved lines and has a circular shape. While a curved lines and a circular shape may allow more uniform distribution of the heater 3408, the deposition of curved lines to form a circular heater 3408 may be more challenging than when the heater includes lines at right angles (e.g., like the heater 3508) or at acute angles (e.g., like the heater 3608).

FIG. 35 illustrates a top or bottom view of a heater 3508 (which may be the same as the heaters 108, 208, or 308) in a CMUT (which may be the same as the CMUT 100, 200, or 300), in accordance with certain embodiments described herein. The heater 3508 is disposed on a surface 3552. The surface 3552 may be at the bottom of the cavity in a CMUT or at the top of the cavity in a CMUT. For example, the surface 3552 may be above an electrode (e.g., the electrode 102) or a CMUT or on a membrane (e.g., the membrane 116) of a CMUT. In embodiments in which the surface 3552 is above the electrode, the surface may be a top surface of the oxide layer 106 or the oxide layer 104, and the view in FIG. 35 may be a top view. In embodiments in which the surface 3552 is on a membrane, the surface may be a bottom surface of the oxide layer 112 and the view in FIG. 35 may be a bottom view. The heater 3508 includes lines at right angles. Deposition of a heater 3508 with lines at right angles may be less challenging than a heater with curved lines in a circular shape (e.g., the heater 3408).

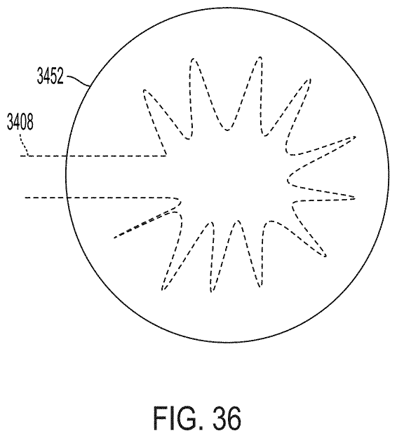

FIG. 36 illustrates a top or bottom view of a heater 3608 (which may be the same as the heaters 108, 208, or 308) in a CMUT (which may be the same as the CMUT 100, 200, or 300), in accordance with certain embodiments described herein. The heater 3608 is disposed on a surface 3652. The surface 3652 may be at the bottom of the cavity in a CMUT or at the top of the cavity in a CMUT. For example, the surface 3652 may be above an electrode (e.g., the electrode 102) or a CMUT or on a membrane (e.g., the membrane 116) of a CMUT. In embodiments in which the surface 3652 is above the electrode, the surface may be a top surface of the oxide layer 106 or the oxide layer 104, and the view in FIG. 36 may be a top view. In embodiments in which the surface 3652 is on a membrane, the surface may be a bottom surface of the oxide layer 112 and the view in FIG. 36 may be a bottom view. The heater 3608 includes lines at acute angles. Deposition of a heater 3608 with lines at acute angles may be less challenging than a heater with curved lines in a circular shape (e.g., the heater 3408).

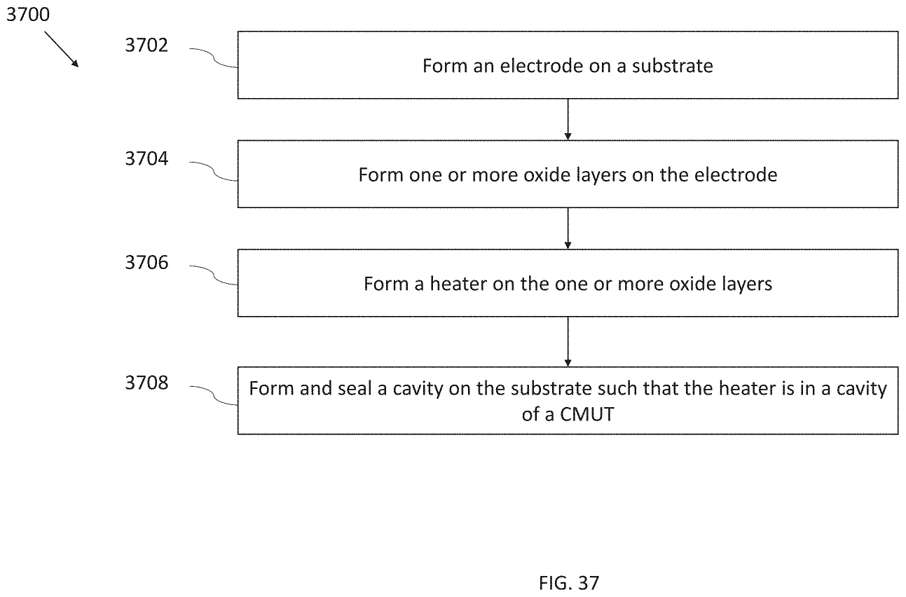

FIG. 37 illustrates a process 3700 for fabricating a CMUT (e.g., the CMUT 100) with a heater (e.g., the heater 108) disposed in the cavity (e.g., the cavity 110) of the CMUT, in accordance with certain embodiments described herein. In some embodiments, the heater may be disposed on an electrode of the CMUT at the bottom of the cavity, and in some embodiments, the heater may be disposed adjacent to the cavity. Further description of the process 3700 may be found with reference to FIGS. 4-18.

In act 3702, an electrode (e.g., the electrode 102) is formed on a substrate (e.g., the substrate 418).

In act 3704, one or more oxide layers (e.g., the oxide layer 104 and/or the oxide layer 106) are formed on the electrode 102.

In act 3706, a heater (e.g., the heater 108) is formed on the one or more oxide layers.

In act 3708, a cavity (e.g., the cavity 110) is formed and sealed on the substrate such that the heater is in the cavity. The heater may thus be disposed between the electrode and a membrane of the CMUT.

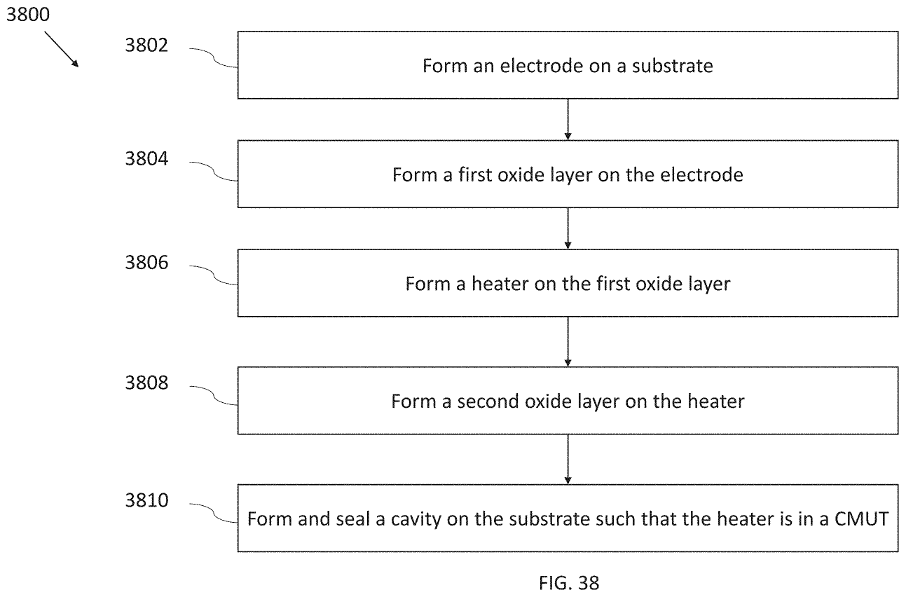

FIG. 38 illustrates a process 3800 for fabricating a CMUT (e.g., the CMUT 200) with a heater (e.g., the heater 208) disposed in the CMUT, in accordance with certain embodiments described herein. In some embodiments, the heater may be disposed on an electrode of the CMUT at the bottom of the cavity. Further description of the process 3800 may be found with reference to FIGS. 19-27.

In act 3802, an electrode (e.g., the electrode 102) is formed on a substrate (e.g., the substrate 418).

In act 3804, a first oxide layer (e.g., the oxide layer 104) is formed on the substrate.

In act 3806, a heater (e.g., the heater 208) is formed on the first oxide layer.

In act 3808, a second oxide layer (e.g., the oxide layer 206) is formed on the heater.

In act 3810, a cavity (e.g., the cavity 210) is formed and sealed on the substrate such that the heater is in the CMUT. The heater may be disposed between the electrode and a membrane of the CMUT.

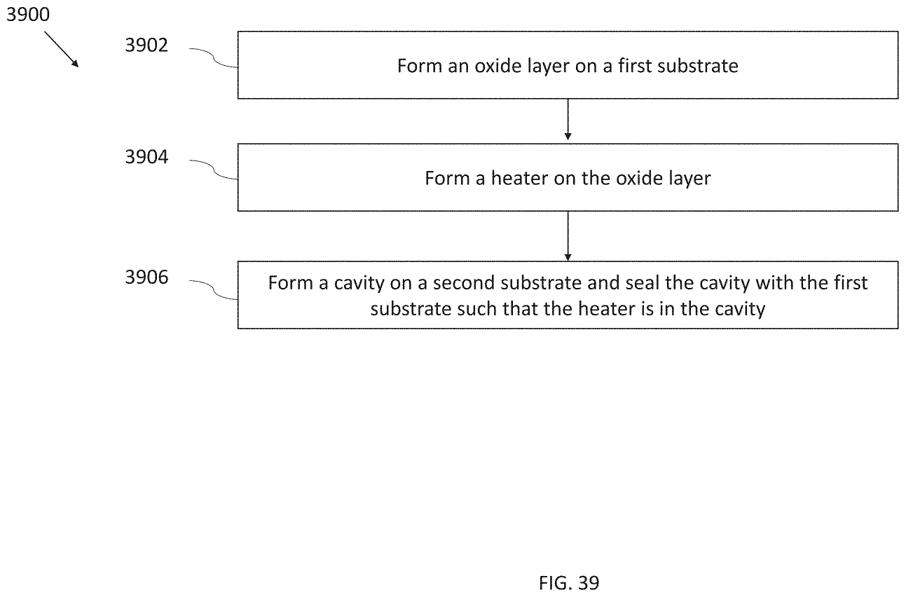

FIG. 39 illustrates a process 3900 for fabricating a CMUT (e.g., the CMUT 300) with a heater (e.g., the heater 308) disposed in the cavity (e.g., the cavity 310) of the CMUT, in accordance with certain embodiments described herein. In some embodiments, the heater may be disposed on the membrane (e.g. the membrane 116) of the CMUT at the top of the cavity of the CMUT. In some embodiments, the heater may be disposed adjacent to the cavity. Further description of the process 3900 may be found with reference to FIGS. 28-33.

In act 3902, an oxide layer (e.g., the oxide layer 112) is formed on a first substrate (e.g., the substrate 1436).

In act 3904, a heater (e.g., the heater 308) is formed on the oxide layer.

In act 3906, a cavity (e.g., the cavity 310) is formed on a second substrate (e.g., the substrate 418) and sealed with the first substrate (e.g., by bonding a layer disposed on the first substrate to a layer disposed on the first substrate) such that the heater is in the cavity. The first substrate may constitute the membrane of the CMUT. The heater may thus be disposed between an electrode and membrane of the CMUT.

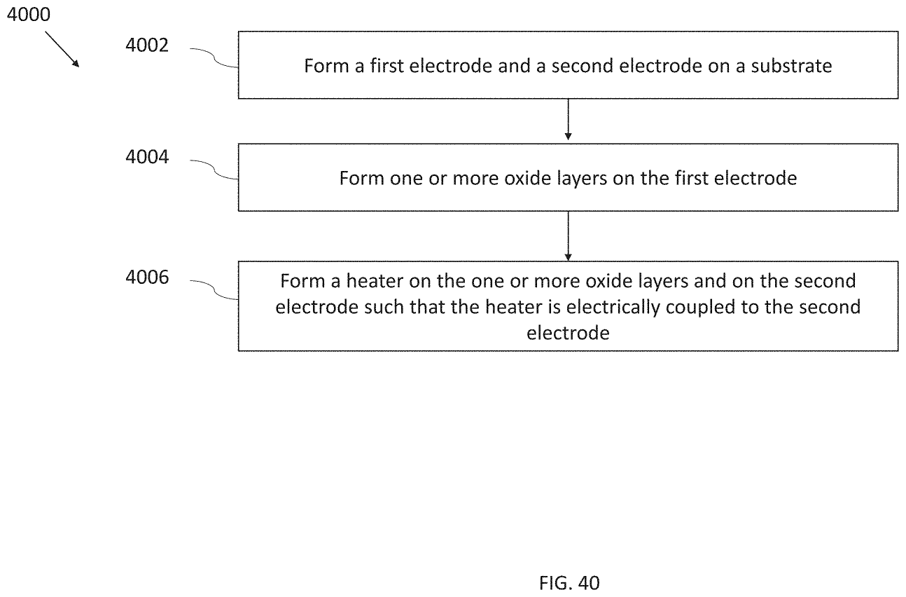

FIG. 40 illustrates a process 4000 for fabricating a CMUT (e.g., the CMUTs 100 or 200) with a heater (e.g., the heaters 108 or 208) disposed in the CMUT, in accordance with certain embodiments described herein. In some embodiments, the heater may be disposed on an electrode of the CMUT at the bottom of the cavity. Further description of the process 4000 may be found with reference to FIGS. 4-27.

In act 4002, a first electrode (e.g., the electrode 102) and a second electrode (e.g., the electrode 102′) are formed on a substrate (e.g., the substrate 418).

In act 4004, one or more oxide layers are formed on the first electrode. For example, two oxide layers (e.g., the oxide layer 104 and the oxide layer 106) may be formed on the first electrode. As another example, one oxide layer (e.g., the oxide layer 104) may be formed on the first electrode. In some embodiments, the one or more oxide layers may be formed on both the first electrode and the second electrode and then etched away from above the second electrode.

In act 4006, a heater (e.g., the heater 108 or 208) is formed on the one or more oxide layers and on the second electrode such that the heater is electrically coupled to the second electrode. Thus, the heater may be disposed on the one or more oxide layers above the first electrode and also disposed on the second electrode. In some embodiments, an oxide layer (e.g., the oxide layer 206) may then be formed on the heater above the first electrode. Integrated circuitry (e.g., the integrated circuitry 421) in the substrate may apply a voltage to the heater through the second electrode. Applying a voltage to the heater may cause the heater to heat. The relationship between voltage applied to the heater and temperature of the heater may be approximately linear.

In some embodiments, the process 4000 may be performed in conjunction with the process 3700. For example, the first electrode formed in act 4002 may be the same as the electrode formed in act 3702, the one of more oxide layers formed in act 4004 may be the same as the one or more oxide layers formed in act 3704, and the heater formed in act 4006 may be the same as the heater formed in act 3706. In some embodiments, the process 4000 may be performed in conjunction with the process 3800. For example, the first electrode formed in act 4002 may be the same as the electrode formed in act 3802, the one of more oxide layers formed in act 4004 may be the same as the first oxide layer formed in act 3804, and the heater formed in act 4006 may be the same as the heater formed in act 3806. The second oxide layer formed in act 3808 may be formed after the act 4006.

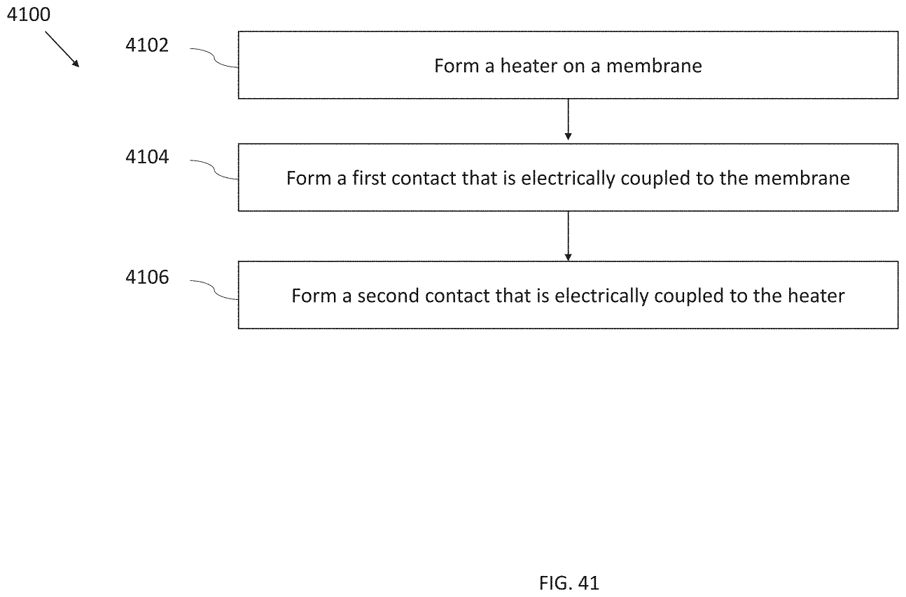

FIG. 41 illustrates a process 4100 for fabricating a CMUT (e.g., the CMUT 300) with a heater (e.g., the heater 308) disposed in the CMUT, in accordance with certain embodiments described herein. In some embodiments, the heater may be disposed on the membrane (e.g., the membrane 116) of the CMUT at the top of the cavity. In some embodiments, the heater may be disposed adjacent to the cavity. Further description of the process 4100 may be found with reference to FIGS. 28-33.

In act 4102, a heater (e.g., the heater 308) is formed on a membrane (e.g., the membrane 116) of a CMUT (e.g., the CMUT 300). In some embodiments, the heater may be formed on a substrate (e.g., the substrate 1436) that, after bonding to another substrate (e.g., the substrate 418), serves as the membrane of the CMUT.

In act 4104, a first contact (e.g., the contact 1850) that is electrically coupled to the membrane is formed.

In act 4106, a second contact (e.g., the contact 3350) that is electrically coupled to the heater is formed. Circuitry in the ultrasound imaging device (e.g., a DC-DC converter such as a charge pump) may apply a voltage to the membrane through the first contact. Circuitry in the ultrasound imaging device (e.g., the DC-DC converter such as the charge pump, or integrated circuitry in an ultrasound-on-chip that includes the CMUT) may also apply a voltage to the heater through the second contact. Applying a voltage to the heater may cause the heater to heat. The relationship between voltage applied to the heater and temperature of the heater may be approximately linear.

In some embodiments, the process 4100 may be performed in conjunction with the process 3900. For example, the heater formed in act 4102 may be the same as the heater formed in act 3904. Acts 4104 and 4106 may be performed after act 3906.

It should be appreciated that the processes for fabricating CMUTs and heaters described with reference to FIGS. 4-41 are non-limiting, and other processes may be used for fabricating CMUTs that include heaters, such as the CMUTs 100, 200, and 300.

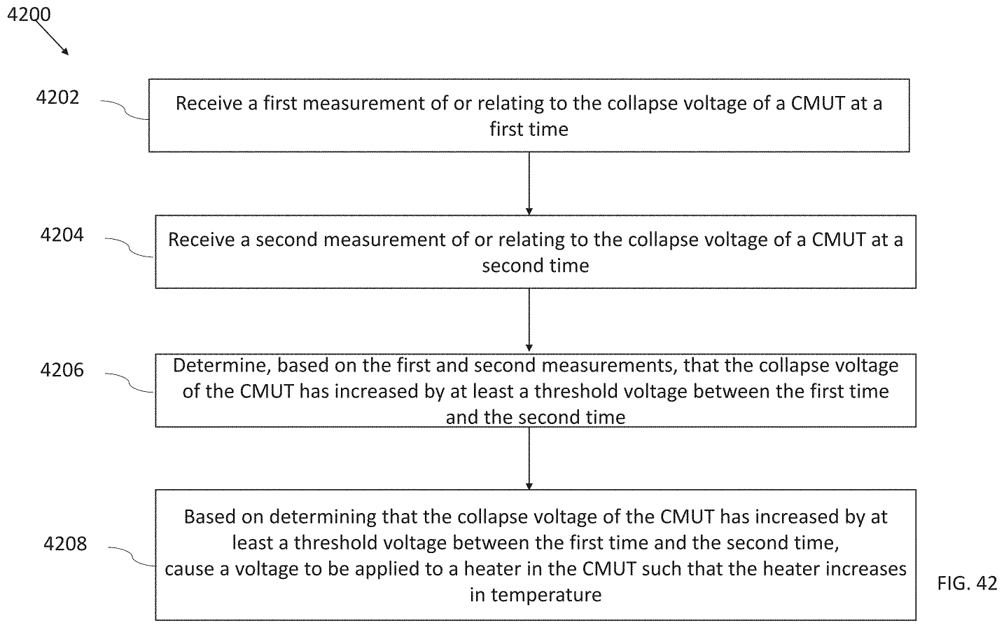

FIG. 42 illustrates a process 4200 for activating a heater (e.g., the heater 108, 208, 308, 3408, 3508, and/or 3608) in a CMUT (e.g., the CMUT 100, 300, or 200) based on the collapse voltage of the CMUT, in accordance with certain embodiments described herein. The process 4200 may be performed by a processing device (e.g., the processing device 4322 described below) in operative communication with an ultrasound imaging device (e.g., the ultrasound imaging device 4302 described below) that contains the CMUT. The processing device may be, for example, a mobile phone, tablet, or laptop. The ultrasound imaging device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

In act 4202, the processing device receives a first measurement of or relating to the collapse voltage of a CMUT (e.g., the CMUT 100, 300, or 200) at a first time. In some embodiments, measurements of or relating to the collapse voltage of the CMUT may be taken after each ultrasound imaging session, and the first time may be after one such ultrasound imaging session. In some embodiments, measurements of or relating to the collapse voltage of the CMUT may be measured after every N ultrasound imaging sessions (where N may be any number such as 2, 3, 4, 5, 6, 7, 8, 9, 10, or any other suitable number), and the first time may be after one such group of N ultrasound imaging sessions. In some embodiments, the ultrasound imaging device may use integrated circuitry (e.g., the integrated circuitry 421) in the ultrasound-on-chip device and/or circuitry external to the ultrasound-on-chip device to perform the first measurement and transmit it to the processing device over a communication link. In some embodiments, the first measurement may be a measurement of collapse voltage. In some embodiments, the first measurement may relate to collapse voltage in that the measurement may be of another parameter that can be used by the processing device to calculate collapse voltage.

In some embodiments, the measurement may include applying a bias voltage VBIAS to the membrane (e.g., the membrane 116) of a CMUT, inputting a constant current Iᵣₐₘₚ to the electrode (e.g., the electrode 102) of the CMUT in order to generate a voltage ramp across the CMUT that begins at a positive voltage supply VDDA and proceeds to ground and/or vice versa, and measuring a time Tramp that it takes for the voltage ramp to exceed a reference voltage value VREF. The capacitance of the CMUT may then be computed as C=Iᵣₐₘₚ×Tᵣₐₘₚ/VDDA−Vref. This procedure may be repeated for multiple values of VBIAS to produce a C v. VBIAS curve. The collapse voltage may be the value of VBIAS at which a discontinuity occurs in this curve. A discontinuity may be detected by computing the derivative (e.g., first or second derivative) of the curve. In some embodiments, the ultrasound imaging device may measure Tramp at different bias voltages and transmit the measurements of Tramp to the processing device. In some embodiments, the ultrasound imaging device may measure Tramp at different bias voltages, compute C based on Tramp, and transmit the C measurements to the processing device. In some embodiments, the ultrasound imaging device may measure Tramp at different bias voltages, compute C based on Tramp, compute the collapse voltage based on the measurements of C, and transmit the collapse voltage measurement to the processing device.

In act 4204, the processing device receives a second measurement of or relating to the collapse voltage of the CMUT at a second time. In embodiments in which measurements of or relating to the collapse voltage of the CMUT are taken after each ultrasound imaging session, the first time may be after one ultrasound imaging session and the second time may be after the immediately subsequent ultrasound imaging session. In embodiments in which measurements of or relating to the collapse voltage of the CMUT are taken after every N ultrasound imaging sessions (where N may be any number such as 2, 3, 4, 5, 6, 7, 8, 9, 10, or any other suitable number), the first time may be after N ultrasound imaging sessions and the second time may be after the subsequent group of N ultrasound imaging sessions. Further description of measurements may be found with reference to act 4202.

In act 4206, the processing device determines, based on the first and second measurements, that the collapse voltage of the CMUT has increased by at least a threshold voltage between the first time and the second time. In embodiments in which the processing device received measurements of another parameter that can be used to calculate collapse voltage, the processing device may calculate the collapse voltage at the first and second times. The threshold voltage may be, for example, 1, 2, 3, 4, 5, 6, 7, 8, 9, 10, 11, 12, 13, 14 V, or any other suitable voltage.

In act 4208, based on determining that the collapse voltage of the CMUT has increased by at least a threshold voltage between the first time and the second time, the processing device may cause a voltage to be applied to a heater (e.g., the heater 108, 308, or 208) in the CMUT such that the heater increases in temperature. In some embodiments, the processing device may transmit a command to the ultrasound imaging device to apply the voltage to the heater. In some embodiments, integrated circuitry (e.g., the integrated circuitry 421) may apply the voltage to the heater (e.g., through the vias 423, the metallization 424, the vias 732, and the electrode 102′). In some embodiments, circuitry external to the ultrasound-on-chip, such as a DC-DC converter such as a charge pump in the ultrasound imaging device, may apply the voltage to the heater (e.g., through the contact 3350). In some embodiments, the voltage applied to the heater may cause the heater to heat to a temperature approximately equal to or between 20-250 degrees Celsius. As specific examples, the temperature may be approximately equal to or between 50-250 degrees, 100-250 degrees, 150-250 degrees, or 200-250 degrees.

In some embodiments, at acts 4202 and 4204, the processing device may receive measurements from multiple CMUTs (e.g., all the CMUTs in the ultrasound imaging device, or a subset thereof) at the first time. In such embodiments, at act 4206, the processing device may determine that the collapse voltage of certain of the CMUTs measured, but not all, has increased by at least the threshold voltage between the first and second times, and at act 4208, the processing device may cause a voltage to be applied to the heaters in those CMUTs to cause those heaters to heat. Alternatively, at act 4206, the processing device may determine that the collapse voltage of certain of the CMUTs measured, but not all, has increased by at least the threshold voltage between the first and second times, and at act 4208, the processing device may cause a voltage to be applied to the heaters in all the CMUTs in the ultrasound imaging device. Alternatively, in some embodiments in which the processing device may receive measurements from multiple CMUTs, at act 106, the processing device may determine if the average of the collapse voltages of all the measured CMUTs has increased by at least the threshold voltage between the first and second times. If so, at act 4208, the processing device may cause a voltage to be applied to the heaters in all the CMUTs in the ultrasound imaging device.

In some embodiments, the first and second measurements may be taken automatically, such that no user input is required to initiate the first and second measurements. In some embodiments, prior to acts 4202 and 4204, the processing device may provide a notification (e.g., on its display screen) that the first and second measurements are being performed (e.g., so that a user of the processing device and/or ultrasound imaging device does not turn either off while the measurement are being performed). In some embodiments, prior to acts 4202 and 4204, the processing device may provide an option to the user whether to proceed with taking the first and second measurements, and a user may select whether to proceed or not.

In some embodiments, in act 4208, the processing device may cause a voltage to be applied to the heater automatically. In some embodiments, prior to act 4208, the processing device may provide a notification (e.g., on its display screen) that the heater will be heated (e.g., so that a user of the processing device and/or ultrasound imaging device does not turn either off during the heating). In some embodiments, prior to act 4208, the processing device may provide an option to the user whether to proceed with causing the voltage to be applied to the heater, and a user may select whether to proceed or not.

As described with reference to FIG. 42, the heater may be activated when in the field (i.e., after being supplied to a user). In some embodiments, the heater may also be activated by a supplier of the ultrasound imaging device prior to supplying the ultrasound imaging device to a user. This may help to prevent CMUTs from becoming stuck early in the lifetime of the ultrasound imaging device. Activating the heater may include applying a voltage to the heater in order to cause the heater to generate heat. In some embodiments, such as ultrasound imaging devices including the CMUTs 100 and 200, the CMUT may include a first electrode (e.g., the electrode 102) and a second electrode (e.g., the electrode 102′), where the second electrode is electrically coupled to the heater (e.g., the heater 108 or 208). The CMUT may be disposed on a substrate (e.g., the substrate 418) that includes integrated circuitry (e.g., the integrated circuitry 421) that is coupled to the second electrode. Applying the voltage to the heater may include using the integrated circuitry to apply the voltage to the heater through the second electrode.

In some embodiments, such as ultrasound imaging devices including the CMUT 300, the CMUT may include a membrane (e.g., the membrane 116) on which the heater (e.g., the heater 308) is disposed. A first electrical contact (e.g., the electrical contact 1850) may be disposed on the membrane and electrically coupled to the membrane. A second electrical contact (e.g., the electrical contact 3350) may be disposed on the membrane and electrically coupled to the heater. In some embodiments, the CMUT may be disposed on a substrate (e.g., the substrate 418) that includes integrated circuitry (e.g., the integrated circuitry 421) that is coupled to the second electrode. Applying the voltage to the heater may include using the integrated circuitry to apply the voltage to the heater through the second electrode. In some embodiments, a DC-DC converter (e.g., external to the ultrasound-on-chip device) may be electrically coupled to the second electrical contact, and applying the voltage to the heater in order to cause the heater to generate heat includes using the DC-DC converter to apply the voltage to the heater through the second electrical contact. Applying a voltage to the heater may cause the heater to heat to a temperature approximately equal to or between 20 to 250 degrees Celsius.

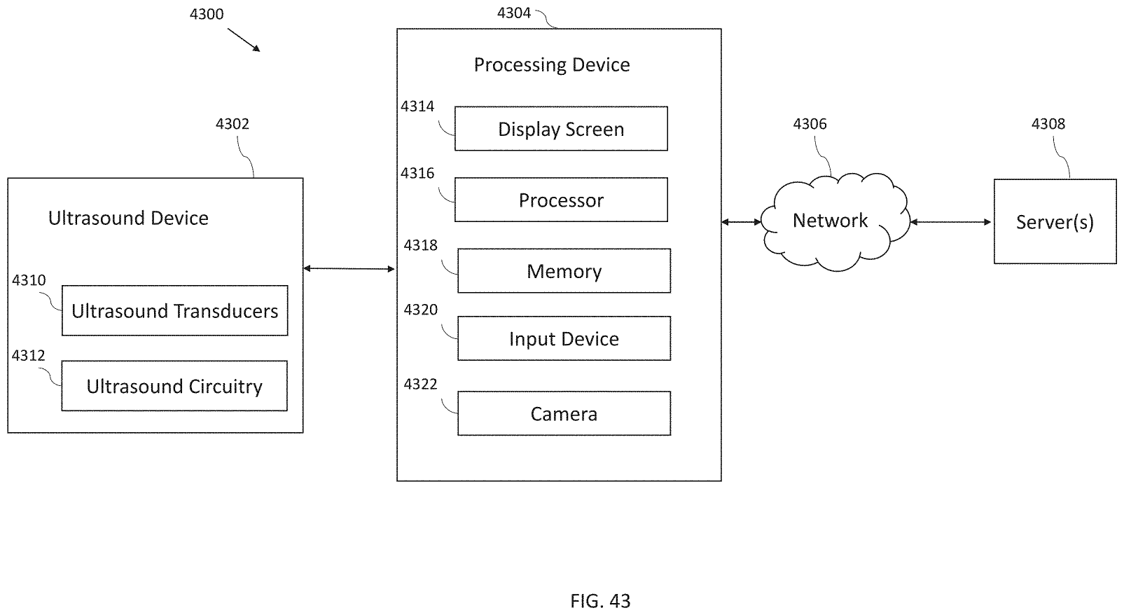

FIG. 43 illustrates a schematic block diagram of an example ultrasound system 4300, in accordance with certain embodiments described herein. The ultrasound system 4300 includes an ultrasound imaging device 4302, a processing device 4304, a network 4306, and one or more servers 4308. The ultrasound imaging device 4302 may be any of the ultrasound imaging devices described herein. The processing device 4304 may be any of the processing devices described herein (e.g., the processing device that performs the process 4200).

The ultrasound imaging device 4302 includes ultrasound transducers 4310 and ultrasound circuitry 4312. The processing device 4304 includes a display screen 4314, a processor 4316, a memory 4318, an input device 4320, and a camera 4322. The processing device 4304 is in wired (e.g., through a lightning connector or a mini-USB connector) and/or wireless communication (e.g., using BLUETOOTH, ZIGBEE, and/or WiFi wireless protocols) with the ultrasound imaging device 4302. The processing device 4304 is in wireless communication with the one or more servers 4308 over the network 4306. However, the wireless communication with the processing device 4308 is optional.

The ultrasound imaging device 4302 may be configured to generate ultrasound data that may be employed to generate an ultrasound image. The ultrasound imaging device 4302 may be constructed in any of a variety of ways. The ultrasound transducers 4310 may be monolithically integrated onto a single semiconductor die (e.g., the substrate 418). The ultrasound transducers 4310 may include, for example, one or more capacitive micromachined ultrasound transducers (CMUTs) (e.g., one or more of the CMUTs 100, 200, and/or 300), one or more piezoelectric micromachined ultrasound transducers (PMUTs), and/or one or more other suitable ultrasound transducer cells. The ultrasound transducers 4310 may include any of the heaters (e.g., the heaters 108, 208, 308, 3408, 3508, and/or 3608) described herein. In some embodiments, the ultrasound transducers 4310 may be arranged in a two-dimensional array. In some embodiments, the ultrasound transducers 4310 may be integrated on the same die (e.g., the substrate 418) as certain other electronic components in the ultrasound circuitry 4312, such as transmit circuitry (which may include one or more waveform generator and/or pulsers), receive circuitry (which may include one or more receive switches, analog processing circuitry, one or more analog-to-digital converters (ADCs), and/or digital processing circuitry), timing and control circuitry, and/or signal conditioning/processing circuitry (which may include multiplexed digital processing circuitry such as image formation circuitry) to form a monolithic ultrasound imaging device. In some embodiments, the ultrasound transducers 4310 and certain components of the ultrasound circuitry 4312 may be integrated on one die (e.g., the substrate 418) and other components of the ultrasound circuitry 4312 may be integrated on another die. In some embodiments, the ultrasound circuitry 4312 may include transmit circuitry that transmits a signal to a transmit beamformer which in turn drives the ultrasound transducers 4310 elements within a transducer array to emit pulsed ultrasonic signals into a structure, such as a patient. The pulsed ultrasonic signals may be back-scattered from structures in the body, such as blood cells or muscular tissue, to produce echoes that return to the ultrasound transducers 4310. These echoes may then be converted into electrical signals by the ultrasound transducers 4310 and the electrical signals are received by receive circuitry in the ultrasound circuitry 4312. The electrical signals representing the received echoes are sent to a receive beamformer in the ultrasound circuitry 4312 that outputs ultrasound data. The ultrasound circuitry 4312 may be the same as the integrated circuitry 421, or the ultrasound circuitry 4312 may be a portion of the integrated circuitry 421, or the integrated circuitry 421 may be a portion of the ultrasound circuitry 4312. The ultrasound circuitry 421 may include a DC-DC converter such as a charge pump. The ultrasound circuitry 421 may be configured to apply a voltage to heaters in the ultrasound transducers 4310 in order to cause the heaters to generate heat. For example, a DC-DC converter that is part of the ultrasound circuitry 4312 (e.g., part of the integrated circuitry 421 or external to the integrated circuitry 421) in a die (e.g., the substrate 418) may be configured to apply a voltage to heaters as described above with reference to FIGS. 18, 27, and/or 33. The ultrasound imaging device 4302 may transmit ultrasound data and/or ultrasound images to the processing device 4304 over a wired (e.g., through a lightning connector or a mini-USB connector) and/or wireless (e.g., using BLUETOOTH, ZIGBEE, and/or WiFi wireless protocols) communication link.

Referring now to the processing device 4304, the processor 4316 may include specially-programmed and/or special-purpose hardware such as an application-specific integrated circuit (ASIC). For example, the processor 4316 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 4304 may be configured to process the ultrasound data received from the ultrasound imaging device 4302 to generate ultrasound images for display on the display screen 4314. The processing may be performed by, for example, the processor 4316. The processor 4316 may also be adapted to control the acquisition of ultrasound data with the ultrasound imaging device 4302. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 4304 may be configured to perform certain of the processes (e.g., the process 4200) described herein using the processor 4316 (e.g., one or more computer hardware processors) and one or more articles of manufacture that include non-transitory computer-readable storage media such as the memory 4318. The processor 4316 may control writing data to and reading data from the memory 4318 in any suitable manner. To perform certain of the processes described herein, the processor 4316 may execute one or more processor-executable instructions stored in one or more non-transitory computer-readable storage media (e.g., the memory 4318), which may serve as non-transitory computer-readable storage media storing processor-executable instructions for execution by the processor 4316. The camera 4322 may be configured to detect light (e.g., visible light) to form an image. The camera 4322 may be on the same face of the processing device 4304 as the display screen 4314. The display screen 4314 may be configured to display images and/or videos, and may be, for example, a liquid crystal display (LCD), a plasma display, and/or an organic light emitting diode (OLED) display on the processing device 4304. The input device 4320 may include one or more devices capable of receiving input from a user and transmitting the input to the processor 4316. For example, the input device 4320 may include a keyboard, a mouse, a microphone, touch-enabled sensors on the display screen 4314, and/or a microphone. The display screen 4314, the input device 4320, the camera 4322, and the speaker 4312 may be communicatively coupled to the processor 4316 and/or under the control of the processor 4316.

It should be appreciated that the processing device 4304 may be implemented in any of a variety of ways. For example, the processing device 4304 may be implemented as a handheld device such as a mobile smartphone or a tablet. Thereby, a user of the ultrasound imaging device 4302 may be able to operate the ultrasound imaging device 4302 with one hand and hold the processing device 4304 with another hand. In other examples, the processing device 4304 may be implemented as a portable device that is not a handheld device, such as a laptop. In yet other examples, the processing device 4304 may be implemented as a stationary device such as a desktop computer. The processing device 4304 may be connected to the network 4306 over a wired connection (e.g., via an Ethernet cable) and/or a wireless connection (e.g., over a WiFi network). The processing device 4304 may thereby communicate with (e.g., transmit data to) the one or more servers 4308 over the network 4306. For example, a party may provide from the server 4308 to the processing device 4304 processor-executable instructions for storing in one or more non-transitory computer-readable storage media (e.g., the memory 4318) which, when executed, may cause the processing device 4304 to perform certain of the processes (e.g., the process 4200) described herein. For further description of ultrasound circuitry, devices, and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the instant application).

FIG. 43 should be understood to be non-limiting. For example, the ultrasound system 4300, the ultrasound imaging device 4302, and/or the processing device 4304 may include fewer or more components than shown.

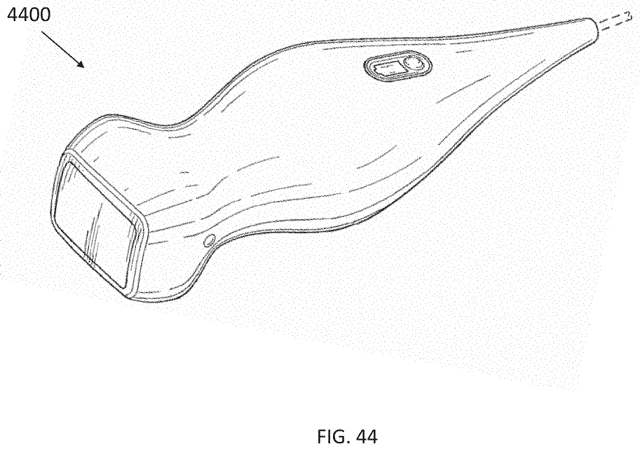

FIG. 44 illustrates an example handheld ultrasound probe 4400, in accordance with certain embodiments described herein. The handheld ultrasound probe 4400 may be the same as the ultrasound imaging device 4302 and may contain ultrasound transducers (e.g., one or more of the CMUT 100, the CMUT 200, the CMUT 300, and/or the ultrasound transducers 4310), any of the heaters (e.g., the heaters 108, 208, 308, 3408, 3508, and/or 3608) described herein, and any or all of the ultrasound circuitry (e.g., the integrated circuitry 421 and/or the ultrasound circuitry 4312) described herein. The ultrasound transducers, the heaters, and some or all of the ultrasound circuitry may be integrated on a die (e.g., the substrate 418) that is contained in the handheld ultrasound probe 4400.

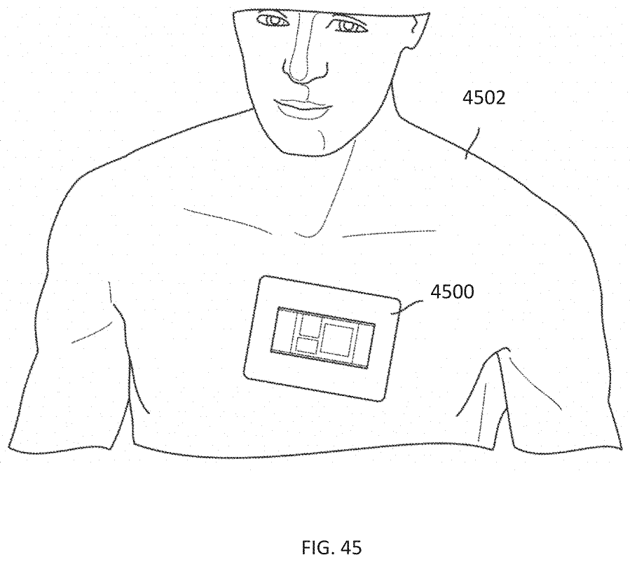

FIG. 45 illustrates an example wearable ultrasound patch 4500, in accordance with certain embodiments described herein. The wearable patch 4500 is coupled to a subject 4502. The wearable ultrasound patch 4500 may be the same as the ultrasound imaging device 4302 and may contain ultrasound transducers (e.g., one or more of the CMUT 100, the CMUT 200, the CMUT 300, and/or the ultrasound transducers 4310), any of the heaters (e.g., the heaters 108, 208, 308, 3408, 3508, and/or 3608) described herein, and any or all of the ultrasound circuitry (e.g., the integrated circuitry 421 and/or the ultrasound circuitry 4312) described herein. The ultrasound transducers, the heaters, and some or all of the ultrasound circuitry may be integrated on a die (e.g., the substrate 418) that is contained in the wearable ultrasound patch 4500.

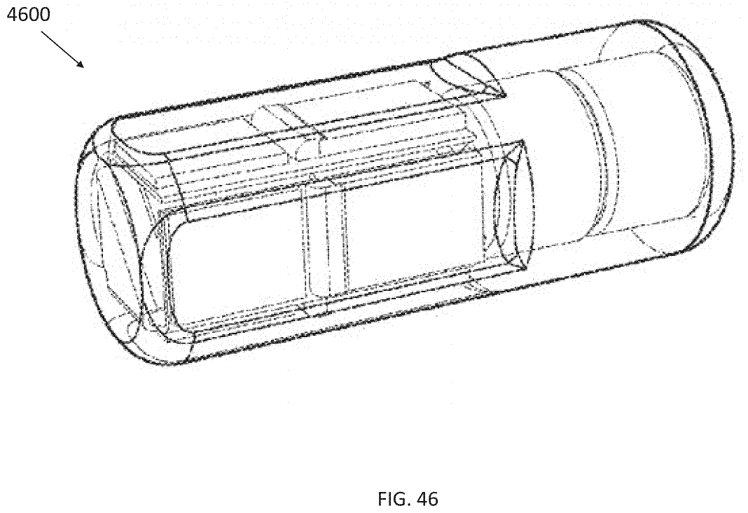

FIG. 46 illustrates an example ingestible ultrasound pill 4600, in accordance with certain embodiments described herein. The ingestible ultrasound pill 4600 may be the same as the ultrasound imaging device 4302 and may contain ultrasound transducers (e.g., one or more of the CMUT 100, the CMUT 200, the CMUT 300, and/or the ultrasound transducers 4310), any of the heaters (e.g., the heaters 108, 208, 308, 3408, 3508, and/or 3608) described herein, and any or all of the ultrasound circuitry (e.g., the integrated circuitry 421 and/or the ultrasound circuitry 4312) described herein. The ultrasound transducers, the heaters, and some or all of the ultrasound circuitry may be integrated on a die (e.g., the substrate 418) that is contained in the ingestible ultrasound pill 4600.

According to an aspect of the present application, an apparatus is provided, comprising a capacitive micromachined ultrasonic transducer (CMUT) and a heater disposed in the CMUT.

In some embodiments, a thickness of the heater is between or equal to approximately 500-1500 angstroms. In some embodiments, the CMUT further comprises an electrode and two or more oxide layers disposed on the electrode, and the heater is disposed between two of the two or more oxide layers, wherein the two or more oxide layers comprise a silicon oxide layer formed using chemical vapor deposition and a silicon oxide layer formed using high-density plasma chemical-vapor deposition, the silicon oxide layer formed using chemical vapor deposition is disposed on the electrode, the heater is disposed on the silicon oxide layer formed using chemical vapor deposition, and the silicon oxide layer formed using high-density plasma chemical-vapor deposition is disposed on the heater.

In some embodiments, the CMUT further comprises a cavity and the silicon oxide layer formed using high-density plasma chemical-vapor deposition is disposed adjacent to the cavity.

In some embodiments, the heater is formed using sputtering or chemical vapor deposition.

In some embodiments, the heater is laid out in a shape that includes curved lines.

In some embodiments, the heater is laid out in a shape that includes lines at right angles.

In some embodiments, the heater is laid out in a shape that includes acute angles.

In some embodiments, the heater is capable of heating to a temperature approximately equal to or between 20 to 250 degrees Celsius.

In some embodiments, the integrated circuitry comprises transmit circuitry, receive circuitry, timing and control circuitry, and/or signal conditioning/processing circuitry.

In some embodiments, the substrate comprises a semiconductor chip.

In some embodiments, the CMUT and the heater disposed therein are disposed on a substrate comprising integrated circuitry.

In some embodiments, the substrate comprises a semiconductor chip.

In some embodiments, the integrated circuitry comprises transmit circuitry, receive circuitry, timing and control circuitry, and/or signal conditioning/processing circuitry.

In some embodiments, the CMUT and the heater disposed therein are disposed in a handheld ultrasound probe.

In some embodiments, the CMUT and the heater disposed therein are disposed in a wearable ultrasound patch.

In some embodiments, the CMUT and the heater disposed therein are disposed in an ingestible ultrasound pill.

According to an aspect of the present application, a method is provided, comprising forming a capacitive micromachined ultrasonic transducer (CMUT) and a heater disposed in the CMUT.

According to some embodiments, the CMUT comprises a membrane and an electrode, and forming the CMUT and the heater disposed in the CMUT comprises forming the heater between the membrane and the electrode of the CMUT.

According to some embodiments, the heater comprises a planar resistive layer.

According to some embodiments, the heater comprises a thin film layer.

According to some embodiments, the heater comprises a layer of nichrome, chromium-silicon, or a ferritic iron-chromium-aluminum alloy.

According to some embodiments, a thickness of the heater is between or equal to approximately 500-3000 angstroms. a thickness of the heater is between or equal to approximately 500-1500 angstroms.

According to some embodiments, the CMUT comprises a cavity having a top and a bottom, and forming the CMUT and the heater disposed in the CMUT comprises forming the heater at the top of the cavity.

According to some embodiments, forming the CMUT and the heater disposed in the CMUT comprises forming an oxide layer on a first substrate, forming a heater on the oxide layer, and forming a cavity on a second substrate and sealing the cavity with the first substrate such that the heater is in the cavity, and wherein the first substrate comprises a membrane on the CMUT.

According to some embodiments, sealing the cavity with the first substrate such that the heater is in the cavity comprises sealing the cavity with the first substrate such that the heater is adjacent to the cavity.

According to some embodiments, the CMUT comprises a cavity having a top and a bottom, and forming the CMUT and the heater disposed in the CMUT comprises forming the heater at the bottom of the cavity.

According to some embodiments, forming the CMUT and the heater disposed in the CMUT comprises forming an electrode on a substrate, forming one or more oxide layers on the electrode, forming a heater on the one or more oxide layers, and forming and sealing a cavity on the substrate such that the heater is in the cavity.

According to some embodiments, sealing the cavity on the substrate such that the heater is in the cavity comprises sealing the cavity on the substrate such that the heater is adjacent to the cavity.

According to some embodiments, forming the one or more oxide layers and forming the heater on the one or more oxide layers comprise forming a silicon oxide layer on the electrode, forming an aluminum oxide layer on the silicon oxide layer, and forming the heater on the aluminum oxide layer.

According to some embodiments, sealing the cavity on the substrate such that the heater is in the cavity comprises sealing the cavity on the substrate such that the heater is adjacent to the cavity.

According to some embodiments, forming the CMUT and the heater disposed in the CMUT comprises forming an electrode on a substrate, forming a first oxide layer on the electrode, forming the heater on the first oxide layer forming a second oxide layer on the heater, and forming and sealing a cavity on the substrate such that the heater is disposed in the CMUT.

According to some embodiments, forming the first oxide layer comprises forming a silicon oxide layer using chemical vapor deposition; and forming the second oxide layer comprises forming a silicon oxide layer using high-density plasma chemical-vapor deposition.

According to some embodiments, sealing the cavity on the substrate comprises sealing the cavity on the substate such that the second oxide layer is adjacent to the cavity.

According to some embodiments, forming the CMUT and the heater disposed in the CMUT comprises: forming an oxide layer in the CMUT, and forming the heater on the oxide layer.

According to some embodiments, forming the CMUT and the heater disposed in the CMUT comprises forming the heater using sputtering or chemical vapor deposition.

According to some embodiments, forming the CMUT and the heater disposed in the CMUT comprises forming the heater in a shape that includes curved lines.

According to some embodiments, forming the CMUT and the heater disposed in the CMUT comprises forming the heater in a shape that includes lines at right angles.

According to some embodiments, forming the CMUT and the heater disposed in the CMUT comprises forming the heater in a shape that includes acute angles.

According to some embodiments, the heater is capable of heating to a temperature approximately equal to or between 20 to 250 degrees Celsius.

According to some embodiments, forming the CMUT and the heater disposed in the CMUT comprises forming a first electrode and a second electrode on a substrate, forming one or more oxide layers on the first electrode, and forming the heater on the one or more oxide layers and on the second electrode such that the heater is electrically coupled to the second electrode.

According to some embodiments, the substrate comprises integrated circuitry, and the integrated circuitry is electrically coupled to the second electrode.

According to some embodiments, the substrate comprises a semiconductor chip.

According to some embodiments, the integrated circuitry comprises transmit circuitry, receive circuitry, timing and control circuitry, and/or signal conditioning/processing circuitry.

According to some embodiments, forming the CMUT and the heater disposed in the CMUT comprises forming the heater on a membrane of the CMUT, forming a first contact that is electrically coupled to the membrane, and forming a second contact that is electrically coupled to the heater.

According to some embodiments, forming the CMUT and the heater disposed in the CMUT comprises forming the CMUT on a substrate comprising integrated circuitry, and wherein the integrated circuitry is electrically coupled to the second electrical contact.

According to some embodiments, forming the CMUT and the heater disposed in the CMUT comprises forming the CMUT on a substrate comprising integrated circuitry.

According to some embodiments, the substrate comprises a semiconductor chip.

According to some embodiments, the integrated circuitry comprises transmit circuitry, receive circuitry, timing and control circuitry, and/or signal conditioning/processing circuitry.

According to an aspect of the present application, a method is provided, comprising applying a voltage to a heater disposed in a CMUT in an ultrasound imaging device in order to cause the heater to generate heat.

In some embodiments, the CMUT includes a first electrode, a second electrode, and one or more oxide layers disposed on the first electrode, the heater is disposed on the one or more oxide layers and on the second electrode, the heater is electrically coupled to the second electrode, the CMUT is disposed on a substrate comprising integrated circuitry, and the integrated circuitry is electrically coupled to the second electrode, and applying the voltage to the heater in order to cause the heater to generate heat comprises using the integrated circuitry to apply the voltage to the heater through the second electrode.

In some embodiments, the integrated circuitry comprises transmit circuitry, receive circuitry, timing and control circuitry, and/or signal conditioning/processing circuitry.

In some embodiments, the substrate comprises a semiconductor chip.

In some embodiments, the CMUT includes a membrane on which the heater is disposed, a first electrical contact disposed on the membrane and electrically coupled to the membrane, and a second electrical contact disposed on the membrane and electrically coupled to the heater, the CMUT is disposed on a substrate comprising integrated circuitry, and the integrated circuitry is electrically coupled to the second electrical contact, and applying the voltage to the heater in order to cause the heater to generate heat comprises using the integrated circuitry is configured to apply a voltage to the heater through the second electrical contact.

In some embodiments, the integrated circuitry comprises transmit circuitry, receive circuitry, timing and control circuitry, and/or signal conditioning/processing circuitry.

In some embodiments, the substrate comprises a semiconductor chip.

In some embodiments, the CMUT includes: a membrane on which the heater is disposed; a first electrical contact disposed on the membrane and electrically coupled to the membrane; and a second electrical contact disposed on the membrane and electrically coupled to the heater; the ultrasound imaging device comprises a DC-DC converter electrically coupled to the second electrical contact; and applying the voltage to the heater in order to cause the heater to generate heat comprises using the DC-DC converter to apply the voltage to the heater through the second electrical contact.

In some embodiments, applying the voltage to the heater in order to cause the heater to generate heat comprises applying a voltage to the heater that causes the heater to heat to a temperature approximately equal to or between 20 to 250 degrees Celsius.

According to an aspect of the present application, a method is provided, comprising receiving, with a processing device in operative communication with an ultrasound imaging device, a first measurement of or relating to a collapse voltage of a CMUT in the ultrasound imaging device at a first time, receiving a second measurement of or relating to the collapse voltage of a CMUT at a second time, determining, based on the first and second measurements, that the collapse voltage of the CMUT has increased by at least a threshold voltage between the first time and the second time, and based on determining that the collapse voltage of the CMUT has increased by at least a threshold voltage between the first time and the second time, automatically causing a voltage to be applied to a heater in the CMUT such that the heater generates heat.

In some embodiments, the first time is after a first ultrasound imaging session and the second time is after a second ultrasound imaging session that is immediately subsequent to the first ultrasound imaging session.

In some embodiments, the first time is after a first ultrasound imaging session and the second time is after a second ultrasound imaging session that is a particular number of ultrasound imaging sessions subsequent to the first ultrasound imaging session.

In some embodiments, the ultrasound imaging device is configured to perform the first and second measurements, and performing the first and second measurements comprises applying a bias voltage to a membrane of the CMUT, inputting a constant current to an electrode of the CMUT such that a voltage ramp is generated across the CMUT, and measuring a time that it takes for the voltage ramp to exceed a reference voltage value, wherein the first and second measurements are performed at different bias voltages.

In some embodiments, receiving the first and second measurements comprises receiving measurements of times that it takes for voltage ramps to exceed the reference voltage value at different bias voltages.

In some embodiments, receiving the first and second measurements comprises receiving measurements of capacitances at different bias voltages.

In some embodiments, the method may further comprise determining the collapse voltage of the CMUT based on detecting a discontinuity in a curve of capacitance versus bias voltage.

In some embodiments, automatically causing the voltage to be applied to a heater in the CMUT such that the heater generates heat comprises transmitting a command to the ultrasound imaging device to apply the voltage to the heater.

Further description of the handheld ultrasound probe 4400, the wearable ultrasound patch 4500, and the ingestible ultrasound pill 4600 may be found in U.S. patent application Ser. No. 15/626,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a capacitive micromachined ultrasonic transducer (CMUT); and
a heater disposed in the CMUT.

2. The apparatus of claim 1, wherein the CMUT comprises a membrane and an electrode, and the heater is disposed between the membrane and the electrode.

3. The apparatus of claim 1, wherein the heater comprises a planar resistive layer.

4. The apparatus of claim 1, wherein the heater comprises a thin film layer.

5. The apparatus of claim 1, wherein the heater comprises a layer of nichrome, chromium-silicon, or a ferritic iron-chromium-aluminum alloy.

6. The apparatus of claim 1, wherein a thickness of the heater is between or equal to approximately 500-3000 angstroms.

7. The apparatus of claim 1, wherein the CMUT comprises a cavity having a top and a bottom, and the heater is disposed at the top of the cavity.

8. The apparatus of claim 1, wherein the CMUT comprises a membrane, the membrane comprises a silicon layer and an oxide layer, and the heater is disposed on the oxide layer.

9. The apparatus of claim 8, wherein the CMUT further comprises a cavity and the heater is disposed adjacent to the cavity.

10. The apparatus of claim 1, wherein the CMUT comprises a cavity having a top and a bottom, and the heater is disposed at the bottom of the cavity.

11. The apparatus of claim 1, wherein the CMUT further comprises an electrode and one or more oxide layers disposed on the electrode, and the heater is disposed on one of the oxide layers.

12. The apparatus of claim 11, wherein the CMUT further comprises a cavity and the heater is disposed adjacent to the cavity.

13. The apparatus of claim 11, wherein the one or more oxide layers comprise a silicon oxide layer and an aluminum oxide layer, the silicon oxide layer is disposed on the electrode, the aluminum oxide layer is disposed on the silicon oxide layer, and the heater is disposed on the aluminum oxide layer.

14. The apparatus of claim 13, wherein the CMUT further comprises a cavity and the heater is disposed adjacent to the cavity.

15. The apparatus of claim 1, wherein the CMUT further comprises an electrode and two or more oxide layers disposed on the electrode, and the heater is disposed between two of the two or more oxide layers.

16. The apparatus of claim 1, wherein the CMUT comprises an oxide layer and the heater is disposed on the oxide layer.

17. The apparatus of claim 1, wherein the CMUT includes:
a first electrode;
a second electrode;
one or more oxide layers disposed on the first electrode; and
a substrate on which the CMUT is disposed, comprising integrated circuitry electrically coupled to the second electrode, wherein the integrated circuitry is configured to apply a voltage to the heater through the second electrode; and
wherein the heater is disposed on the one or more oxide layers and on the second electrode such that the heater is electrically coupled to the second electrode.

18. The apparatus of claim 1, wherein the CMUT includes:
a membrane on which the heater is disposed;
a first electrical contact disposed on the membrane and electrically coupled to the membrane; and
a second electrical contact disposed on the membrane and electrically coupled to the heater.

19. The apparatus of claim 18, wherein the CMUT is disposed on a substrate comprising integrated circuitry, the integrated circuitry is electrically coupled to the second electrical contact, and the integrated circuitry is configured to apply a voltage to the heater through the second electrical contact.

20. The apparatus of claim 18, further comprising a DC-DC converter electrically coupled to the second electrical contact and configured to apply a voltage to the heater through the second electrical contact.
